(19) United States
(12) Patent Application Publication
(10) Pub. No.: US 2017/0121683 A1
(43) Pub. Date: May 4, 2017

(54) ARYL HYDROCARBON RECEPTOR DISRUPTION AND ENHANCEMENT

(71) Applicant: REGENTS OF THE UNIVERSITY OF MINNESOTA, Minneapolis, MN (US)

(72) Inventors: Dan Samuel Kaufman, San Diego, CA (US); Mathew George Angelos, Minneapolis, MN (US)

(21) Appl. No.: 15/342,641

(22) Filed: Nov. 3, 2016

Related U.S. Application Data
(60) Provisional application No. 62/250,210, filed on Nov. 3, 2015.

(51) Int. Cl. C12N 5/0783

(52) U.S. Cl. CPC C12N 5/0646; C12N 2501/999; C12N 2506/02; C12N 2506/45; C12N 2500/32; C12N 2500/44; C12N 2501/115; C12N 2501/165; C12N 2501/125; C12N 2501/2303; C12N 2501/2306; C12N 2501/30; C12N 2501/2315; C12N 2501/2307; C12N 2501/727

(57) ABSTRACT

Methods for enhancing and disrupting activity of the aryl hydrocarbon receptor (AHR) and cells having disrupted or enhanced AHR activity. The methods and cells may be used to enhance production of specific cell populations during hemato-endothelial cell or hemato-lymphoid cell development.

### CONTINUING APPLICATION DATA

This application claims the benefit of U.S. Provisional Application Ser. No. 62/250,210, filed Nov. 3, 2015, which is incorporated by reference herein.

### GOVERNMENT FUNDING

This invention was made with government support under DK107017 and HD060536 awarded by the National Institutes of Health. The government has certain rights in the invention.

### SEQUENCE LISTING

This application contains a Sequence Listing electronically submitted to the United States Patent and Trademark Office via EFS-Web as an ASCII text file entitled “110.05180101_SequenceListing_ST25.txt” having a size of 8 kilobytes and created on Oct. 31, 2016. Due to the electronic filing of the Sequence Listing, the electronically submitted Sequence Listing serves as both the paper copy required by 37 CFR §1.821(c) and the CRF required by §1.821(e). The information contained in the Sequence Listing is incorporated by reference herein.

### SUMMARY

This disclosure provides methods for enhancing and disrupting the activity of the aryl hydrocarbon receptor (AHR), an evolutionarily conserved transcription factor, in a cell. This disclosure further provides a cell having disrupted or enhanced AHR activity. As further described herein, disruption or enhancement of AHR activity during hemato-endothelial cell development, hematopoietic progenitor cell development, and/or hemato-lymphoid cell development may be used to enhance production of specific cell populations.

In one aspect, this disclosure describes a method that includes inducing hematopoietic differentiation of a stem cell and disrupting AHR activity in the cell. In some embodiments, disrupting AHR activity includes treating the cell with an aryl hydrocarbon receptor (AHR) antagonist. In some embodiments, the AHR antagonist includes StemReginin-1 (SR-1), GNF351, and/or 6,2,4-trimethoxyflavone.

In some embodiments, disrupting AHR activity includes reducing AHR activity in the cell. In some embodiments, reducing AHR activity includes reducing expression AHR in a cell. In some embodiments, the method includes completely eliminating expression of AHR. The reduction of expression of AHR can be inducible. In some embodiments, at least a portion of an AHR coding region is eliminated from the cell. In some embodiments, at least a portion of an AHR promoter region is eliminated from the cell.

In some embodiments, the stem cell is an embryonic stem cell including, for example, an H9 human embryonic stem cell (hESC). In some embodiments, the stem cell is an induced pluripotent stem cell (iPSC). In some embodiments, the stem cell is preferably a human cell.

In some embodiments, differentiation of the stem cell (including, for example, an hESC and/or iPSC) to a hemato-endothelial cell, a hematopoietic progenitor cell, and/or a hemato-lymphoid cell is enhanced compared to differentiation without disruption of AHR activity.

A hemato-endothelial cell can include, for example, an endothelial cell (EC), a CD34⁺CD31⁺ cell, and/or a CD34⁺CD144⁺ cell. A hematopoietic progenitor cell can include, for example, a CD34⁺CD43⁺ cell, and/or a CD34⁺CD45⁺ cell. A hemato-lymphoid cell can include, for example, a natural killer cell progenitor (NK progenitor, also referred to herein as NKP) cell and/or a conventional natural killer (cNK) cell. An NK progenitor cell can include, for example, a CD94⁺CD117⁺ cell. A cNK cell can include, for example, a CD94⁺CD117⁻CD56⁺LFA1⁻ cell.

In some embodiments, differentiation of the stem cell to an NK progenitor cell differentiation and/or cNK cell is enhanced compared to differentiation without disruption of AHR activity.

In some embodiments, the method includes treating the cell to enhance hemato-endothelial cell differentiation, hematopoietic progenitor cell differentiation, and/or hemato-lymphoid cell differentiation. In some embodiments, the method includes treating the cell to enhance NK progenitor cell differentiation and/or cNK cell differentiation. The cell can be treated with hemato-endothelial induction medium which may include, for example, at least one of vascular endothelial growth factor, stem cell factor, interleukin (IL)-3, IL-6, and thrombopoietin. The cell can be treated with NK cell differentiation medium which may include, for example, at least one of IL-15, IL-7, Flt-3 ligand, stem cell factor, and IL-3.

This disclosure further describes a method that includes inducing hematopoietic differentiation of a stem cell and enhancing AHR activity in the cell. In some embodiments, enhancing AHR activity includes treating the cell with an AHR agonist. In some embodiments, the AHR agonist includes tetrachlorodibenzo-p-dioxin (TCDD), 6-FICZ (6-Formylindolo (3,2-b)carbazole), ITE (2-(1H-Indol-3-ylcarbonyl)-4-thiazolecarboxylic acid methyl ester), meBIO ((2′Z,3′E)-6-Bromo-1-methylindirubin-3′-oxime), and/or L-Kynurenine.

In some embodiments, enhancing AHR activity includes increasing AHR activity in the cell. In some embodiments, increasing AHR activity includes increasing expression AHR in a cell. In some embodiments, at least a portion of either an AHR coding region or an AHR promoter region or both is mutated. The enhancement of activity and/or expression of AHR can be inducible.

In some embodiments, the stem cell is an embryonic stem cell including, for example, an H9 human embryonic stem cell (hESC). In some embodiments, the stem cell is an induced pluripotent stem cell (iPSC). In some embodiments, the stem cell is preferably a human cell.

In some embodiments, differentiation of the cell to an innate lymphoid cell group 3 (ILC3, also referred to herein as Group 3 ILC) cell is enhanced compared to differentiation without enhancement of AHR activity. In some embodiments, the method includes treating the cell to induce ILC3 cell differentiation. In some embodiments, the cell can be treated with hemato-endothelial induction medium which may include, for example, at least one of vascular endothelial growth factor, stem cell factor, IL-3, IL-6, and thrombopoietin. In some embodiments, the cell can be treated with NK cell differentiation medium which may include, for example, at least one of IL-15, IL-7, Flt-3 ligand, stem cell factor, and IL-3. In some embodiments, the ILC3 cell is at least one of CD94⁻, CD117⁺, CD56⁺, and LFA1⁻. In some embodiments, the ILC3 cell can express at least one of RORc, IL1-R1, and IL-22. In some embodiments, the ILC3 cell can express lower levels of at least one of GATA3 and TBX21/TBET compared to the stem cell.

This disclosure further describes cells derived according to the methods described herein.

This disclosure also describes a cell being homozygous or heterozygous for an AHR mutation. In some embodiments, the cell is a stem cell including, for example, an embryonic stem cell or an induced pluripotent stem cell. In some embodiments, the cell is a hemato-endothelial cell, a hematopoietic progenitor cell, and/or a hemato-lymphoid cell. In some embodiments, the cell is an endothelial cell (EC). In some embodiments, the cell is an NK progenitor cell and/or a cNK cell. In some embodiments, the cell is an ILC3 cell. In some embodiments, the cell is preferably a human cell. The mutation can include a mutation in an AHR coding region.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.).

For any method disclosed herein that includes discrete steps, the steps may be conducted in any feasible order. And, as appropriate, any combination of two or more steps may be conducted simultaneously.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1 shows StemReginin-1 (SR-1), an aryl hydrocarbon receptor (AHR) antagonist, and 2,3,7,8-tetrachlorodibenzo-p-dioxin (TCDD), an AHR agonist, modulate AHR-target genes in H9 human embryonic stem cells (hESCs). Undifferentiated H9 hESCs were treated with either DMSO (vehicle), 1 μM SR-1, or 10 nM TCDD for 96 hours in hESC growth media. Quantitative polymerase chain reaction analysis for AHR and two downstream effector gene targets (CYP1A1 and CYP1B1) are shown. SR-1-treated H9 hESCs yield a reduced expression of both CYP1A1 and CYP1B1 as compared to DMSO controls, while TCDD-treated H9 hESCs yield a significantly increased expression of both CYP1A1 and CYP1B1. p<0.05 as compared to DMSO-treated H9 hESCs (Student's t-test); data were normalized to GAPDH expression and represent an average of three biological replicates with error bars representing standard error of the mean (SEM).

FIG. 2 shows AHR and downstream gene target expression varies over the course of hESC hemato-endothelial differentiation. H9 hESCs were permitted to differentiate as spin embryoid bodies (spin-EBs) exposed to hemato-endothelial induction media. EBs were harvested and disaggregated on days 9, 11, 13, and 15, and total RNA was obtained. Quantitative polymerase chain reaction analysis for AHR and two downstream effector gene targets (CYP1A1 and CYP1B1) demonstrate a significant increase in expression by day 11. *, †, ‡ p<0.05 for CYP1B1, CYP1A1, and AHR, respectively, as compared to undifferentiated H9 hESCs for each gene (two-way ANOVA); data were normalized to GAPDH expression and represent an average of three biological replicates with error bars representing SEM.

FIG. 3 shows AHR antagonism increases CD34⁺CD31⁺ and CD34⁺CD45⁺ early hemato-endothelial and hematopoietic progenitor cell differentiation from hESCs. H9 hESCs were differentiated as spin EB exposed to Stage I mesodermal induction media for 6 days and transferred to hemato-endothelial induction media with either DMSO, 1 μM SR-1, or 10 nM TCDD treatment for an additional 3 days. Non-adherent and adherent cell fractions were harvested, combined, and probed for hemato-endothelial surface antigen expression by flow cytometry. Representative flow cytometry plots are shown from three biologically independent experiments.

FIG. 4 shows AHR agonism attenuates hemato-endothelial progenitor cell persistence from hESCs. H9 hESCs were differentiated as spin EB exposed to Stage I mesodermal induction media for 6 days and transferred to hemato-endothelial induction media with either DMSO, 1 μM SR-1, or 10 nM TCDD treatment for an additional 9 days. Non-adherent and adherent cell fractions were harvested, combined, and probed for hemato-endothelial surface antigen expression by flow cytometry. Representative flow cytometry plots are shown from three biologically independent experiments.

FIG. 5(A-B) shows AHR activity influences the number of early hematopoietic progenitor cells derived from hESCs as quantified by hematopoietic colony-forming cell assay. FIG. 5A. H9 hESCs were differentiated as spin EB exposed to Stage I mesodermal induction media for 6 days and transferred to hemato-endothelial induction media with either DMSO, 1 μM SR-1, or 10 nM TCDD treatment for an additional 6 days. On day 12 of total differentiation, 50,000 non-adherent cells were obtained in each treatment group and seeded in Methocult H4436. Following 14 days of growth, colony-forming units (CFUs) were quantified and scored based on morphology. Data are represented as number of CFUs per 5×10⁴ cells. FIG. 5B. Distribution of CFU morphology for DMSO-, SR-1-, and TCDD-treated H9 hESCs. *p<0.05 as compared to DMSO-treated H9 hESC (Student's t-test) and represent an average of three biological replicates with error bars representing SEM. CFU-GEMM: Colony-Forming Unit Granulocyte/Erythrocyte/Macrophage/Megakaryocyte; CFU-M: Colony-Forming Unit Macrophage; CFU-GM: Colony-Forming Unit-Granulocyte/Macrophage; CFU-E: Colony-Forming Unit Erythroid; BFU-E: Burst-Forming Unit-Erythroid.

FIG. 6 shows AHR antagonism promotes NK cell production from hESCs. H9 hESCs were differentiated as spin EB exposed to Stage I mesodermal induction media for 11 days and transferred to NK-cell differentiation media with either DMSO, 1 μM SR-1, or 10 nM TCDD treatment for an additional 14 and 21 days. At the indicated time points, non-adherent cells were harvested and analyzed for NK-cell immunophenotype (CD56⁺CD45⁺) by flow cytometry. Representative flow cytometry plots are shown from two biologically independent experiments.

FIG. 7(A-C) shows small molecule antagonism of AHR enhances early hemato-endothelial development from hESCs. FIG. 7A. Schema of hESC differentiation into early hemato-endothelial cells as spin-embryoid bodies (spin-EBs). hESCs are made into spin-EBs at Day 0 and conditioned into mesoderm lineages for 6 days using defined cytokines (Stage 1). At Day 6, spin-EBs are transferred into hemato-endothelial culture media (Stage 2) to promote endothelial and hematopoietic cell differentiation. For these studies, cells are treated with either 1 μM SR-1, 10 nM TCDD, or DMSO vehicle control beginning at Day 6+0 with media exchanges and/or harvesting performed at Day 6+3, Day 6+6, and Day 6+9. FIG. 7B. Representative flow cytometry plots of one hESCs differentiation. Both adherent and non-adherent cell fractions are harvested at Day 6+3, Day 6+6, and Day 6+9 and assessed for endothelial cell (CD34⁺CD31⁺, CD34⁺CD144⁺) and hematopoietic progenitor cell (CD34⁺CD43⁺, CD34⁺CD45⁺) phenotypes. FIG. 7C. Fold change of the total percentage of each hemato-endothelial phenotype for SR-1 and TCDD treated hESCs normalized to matched DMSO treated controls. n=4-6, error bars represent SEM, *p<0.05 as compared to DMSO treated controls by student's t-test. N/A: Not applicable due to absence appreciable of CD34⁺CD45⁺ populations at Day 6+3 time point.

FIG. 8(A-C) shows AHR is implicated in normal human hematopoiesis and can be targeted by small molecules in hESCs. FIG. 8A. Non-adherent hematopoietic progenitor cells derived from hESCs were harvested at Day 6+3, Day 6+5, Day 6+7, and Day 6+9 time points and probed for gene expression by quantitative real-time PCR (qPCR). For each gene, Cₜ values were normalized to GAPDH at each time point and data is presented as relative fold-change as compared to undifferentiated hESCs. Differentiation was confirmed by a significant reduction of OCT4, a marker of pluripotency. n=3, error bars represent SEM, *p<0.05, **p<0.01, ***p<0.001, ****p<0.0001 using student's t-test. FIG. 8B Undifferentiated hESCs were cultured in the presence of 1 μM SR-1, 10 nM TCDD, or DMSO vehicle control for 96 hours and probed for gene expression by qPCR. For each gene, Cₜ values were normalized to GAPDH at each time point and data is presented as relative fold-change as compared to DMSO treated hESCs. n=3, error bars represent SEM, *p<0.05 assessed by two-way ANOVA+Tukey-Kramer multiple comparisons post-hoc test. FIG. 8C. Both adherent and non-adherent cell fractions treated with either DMSO, SR-1, or TCDD were harvested at Day 6+3, Day 6+6, and Day 6+9 and probed for viability with Sytox Blue Live/Dead Stain. Total percentage of viable cells for each group and time point are plotted. n=3.

FIG. 9(A-B) shows SR-1 treated hESCs demonstrate increased multilineage hematopoietic development. FIG. 9A. Non-adherent hematopoietic progenitor cells derived from hESCs differentiated in the presence of SR-1, TCDD, or DMSO controls were harvested at Day 6+5 and seeded at 50,000 cells/dish in a standard methylcellulose colony-forming unit assay (CFU). Colonies were counted for each treatment group following 2 weeks of culture and scored for the following morphological subsets: BFU-E: burst-forming unit-erythroid; CFU-E: colony-forming unit-erythroid; CFU-GM: colony-forming unit-granulocyte, macrophage, CFU-M: colony-forming unit-macrophage; CFU-GEMM: colony-forming unit-granulocyte, erythroid, macrophage, megakaryocyte. n=3, error bars represent SEM of the total number of colonies/50,000 cells seeded, *p<0.05 as assessed with one-way ANOVA+Tukey-Kramer multiple comparisons post-hoc test. FIG. 9B. Non-adherent hematopoietic progenitor cells derived from hESCs differentiated in the presence of SR-1, TCDD, or DMSO controls were harvested at Day 6+3, Day 6+6, and Day 6+9 time points and probed for gene expression by quantitative real-time PCR (qRT-PCR). For each gene, Cₜ values were normalized to GAPDH at each time point and data is presented as relative fold-change to DMSO treated controls. n=3, error bars represent SEM, *p<0.05, **p<0.01 using student's t-test.

FIG. 10(A-B) shows AHR regulates cell cycle progression during hematopoietic cell development from hESCs. FIG. 10A. Non-adherent hematopoietic progenitor cells derived from hESCs were cultured in the presence of DMSO, SR-1, and TCDD until Day 6+5. Cell cycle phase was assessed using flow cytometry to determine the total percentage of G₀/G₁)(EdU⁻PI^{lo}, S-phase (EdU⁺PI⁺), or G₂/M (EdU⁻PI^{hi}). Representative flow cytometry plots are shown. FIG. 10B. Quantification of cell cycle analysis, n=3, *p<0.05, **p<0.01, ***p<0.001 as assessed by two-way ANOVA+Tukey-Kramer multiple comparisons post-hoc test.

FIG. 11 (A-F) shows CRISPR/Cas9 engineered hESCs with AHR deletion demonstrate increased early hemato-endothelial cell development. FIG. 11A. gRNA cassette design targeting AHR. IS: insertion sequence; U6: Promoter, gRNA Exon 1: 22-nt gRNA specific to AHR exon 1, Term: termination sequence. FIG. 11B. Gel electrophoresis of PCR products produced from clonally-derived hESC-RUNx1c-tdTomato cells nucleofected with AHR gRNA cassette. Genomic DNA was harvested and primers flanking the AHR exon 1 locus were used to generate a PCR product with predicted full-length of 718 bp. WT: Negatively selected nucleofected hESC-RUNX1c-tdTomato hESCs; +/−: Individual clones with AHR heterozygous deletion (AHR^{+/-}); −/−: Individual clones with AHR homozygous deletion (AHR^{-/-}); *: 718 bp amplicon, ̂: 571 bp amplicon. FIG. 11C. Immunoblot of protein lysate harvested from K562 cells (positive control), NK92 natural killer cells (positive control), wild-type hESC-RUNX1c-tdTomato (hESC-R1c-tdTom), heterozygous AHR deleted hESC-RUNX1c-tdTomato (+/−), and homozygous AHR deleted hESC-RUNX1c-tdTomato (−/−). AHRR: aryl hydrocarbon receptor repressor. FIG. 11D. Representative flow cytometry plots at Day 6+3, Day 6+6, and Day 6+9 from one differentiation of wild-type hESC-RUNX1c-tdTomato (WT), heterozygous AHR hESC-RUNX1c-tdTomato deletion (AHR^{+/-}), and homozygous AHR hESC-RUNX1c-tdTomato deletion (AHR^{-/-}). Both adherent and non-adherent cell fractions are harvested at Day 6+3, Day 6+6, and Day 6+9 and assessed for endothelial (CD31, CD144), and hematopoietic (CD33, CD41a, CD43, CD45) phenotype. FIG. 11E. Representative flow cytometry plots at Day 6+3 and Day 6+6 from one differentiation assessing for RUNX1c expression based on tdTomato fluorescent reporter protein. FIG. 11F. Non-adherent hematopoietic progenitor cells derived from WT hESC-RUNX1c-tdTomato, AHR^{+/-} hESC-RUNX1c-tdTomato, or AHR^{-/-} hESC-RUNX1c-tdTomato were harvested at Day 6+5 and seeded at 50,000 cells/dish in a standard methylcellulose colony-forming unit assay (CFU). Colonies were counted for each treatment group following 2 weeks of culture and scored for the following morphological subsets, as previously described. n=3, error bars represent SEM of the total number of colonies/50,000 cells seeded, *p<0.05 as assessed with one-way ANOVA+Tukey-Kramer multiple comparisons post-hoc test.

FIG. 12(A-B) shows CRISPR/Cas9 engineered hESCs with AHR deletion demonstrate increased early hemato-endothelial cell development. FIG. 12A. Quantification from flow cytometry profiles at Day 6+3, Day 6+6, and Day 6+9 of CD34⁺CD144⁺ and CD34⁺CD31⁺ endothelial cells and CD34⁺CD43⁺ and CD34⁺CD45⁺ early hematopoietic progenitor cells differentiated from wild-type hESC-RUNX1c-tdTomato (WT) or AHR^{-/-} hESC-RUNX1c-tdTomato (AHR^{-/-}). Total percentage of each cell population was normalized to WT and plotted as fold-change relative to WT. n=3 representative differentiations, *p<0.05 using student's t-test. FIG. 12B. Quantification from flow cytometry profiles at Day 6+3 and Day 6+6 of tdTom⁺CD43⁺ and tdTom⁺CD45⁺ cells derived from WT or AHR^{-/-} hESCs. n=3 representative differentiations, *p<0.05 using student's t-test.

FIG. 13(A-F) shows hESCs differentiated in the presence of SR-1 promotes the development of functional natural killer (NK) cells. FIG. 13A. Schema of hESC differentiation into lymphoid cells as spin-embryoid bodies (spin-EBs). hESCs are made into spin-EBs at Day 0 and cultured in Stage 1 conditions with defined cytokines to promote mesoderm development for 11 days. At Day 11, spin-EBs are transferred onto OP9-DL1 in the presence of NK cell differentiation media (NKDM) to promote lymphoid differentiation. Cells are treated beginning at Day 11+0 with either 1 μM SR-1, 10 nM TCDD, or DMSO vehicle control with media exchanges and/or harvesting performed every week for up to 4 weeks. FIG. 13B. At Day 11, differentiated spin-EBs (photo) are phenotyped for CD34⁺CD45⁺ expression and transferred to OP9-DL1 stroma in NKDM. Non-adherent hematopoietic cells cultured either in the presence of DMSO, SR-1, or TCDD were assessed for developing NK cell immunophenotype based on CD56⁺CD45⁺ expression at Days 11+21, and 11+28; representative flow cytometry plots from one differentiation are shown. FIG. 13C. Quantification of fold-change in total percentage of CD56⁺CD45⁺ cells at both Day 11+21 and Day 11+28 when treated with DMSO, SR-1, or TCDD. SR-1 and TCDD treatments for each differentiation are normalized to DMSO controls. n=3 independent differentiation experiments, error bars represent SEM, *p<0.05 as assessed with two-way ANOVA+Tukey-Kramer multiple comparisons post-hoc test. FIG. 13D. Non-adherent hematopoietic progenitor cells derived from hESCs differentiated in the presence of SR-1, TCDD, or DMSO controls were harvested at Day 11+28 and probed for gene expression by quantitative real-time PCR (qRT-PCR). For each gene, Cₜ values were normalized to GAPDH at each time point and data is presented as relative fold-change to DMSO treated controls. n=3, error bars represent SEM, *p<0.05, #p<0.01 using student's t-test. FIG. 13E. Non-adherent hematopoietic progenitor cells derived from hESCs differentiated in the presence of SR-1, TCDD, or DMSO controls were harvested at Day 11+28 and assessed for CD107a expression following 4 hours of co-culture with K562 target cells at 2:1 effector:target ratio. SSC: side scatter. Representative flow cytometry plots are shown from one experiment. FIG. 13F. Quantification of percentage of CD107a⁺ cells when treated with DMSO, SR-1, or TCDD at Day 11+28, n=2-3 replicates.

FIG. 14(A-B) shows AHR hyperactivation in hESC-derived hematopoietic cells increases Group 3 ILC development. FIG. 14A. Day 11+28 hESC-derived non-adherent hematopoietic cells were treated with SR-1 to enrich for CD94⁺CD117⁻ (cNK), TCDD to enrich for CD94⁻CD117⁺LFA1⁻ (ILC), and DMSO to enrich for CD94⁺CD117⁺ (NKP). Each phenotype was FACS sorted and RNA was harvested to assess for gene expression. *: Sorted population. FIG. 14B. qRT-PCR analysis of genes associated with cNK and ILC3 development. Data are represented as fold-change in gene expression relative to NKP sorted cells. n=2, means of duplicate experiments are shown.

FIG. 15(A-E) shows hESCs differentiated in the presence of SR-1 skews development towards conventional NK cells (cNK) while TCDD supports of the development of an innate lymphoid cell (ILC) phenotype. FIG. 15A. Gating scheme for identifying cNK (CD94⁻CD117⁺), NK progenitor cells (NKP, CD94⁺CD117⁻ and CD94⁺CD117⁻LFA1⁺), and ILC (CD94⁻CD117⁺LFA1⁻) phenotypes. FIG. 15B. Representative flow cytometry profile of non-adherent hematopoietic cells differentiated from hESCs in the presence of DMSO, SR-1, or TCDD at Day 11+28. FIG. 15C. cNK, NKP, and NKP/ILC subpopulations from Day 11+28 DMSO, SR-1, and TCDD differentiated hESCs assessed for CD56 and LFA (CD11a/CD18) surface antigen expression. Representative flow cytometry plots are shown, n=3. FIG. 15D. Total percentage of cNK, NKP, and NKP/ILCs present in the non-adherent fraction of differentiating hESCs in the presence of DMSO, SR-1, or TCDD at Day 11+28. n=3, error bars represent SEM, *p<0.05, **p<0.01, ***p<0.001 as compared to DMSO treated controls and assessed by two-way ANOVA+Tukey-Kramer multiple comparisons post-hoc test. FIG. 15E. CD94⁻CD117⁺ subpopulations were further quantified for expression of LFA⁺ (NKP) and LFA⁻ (ILC) by flow cytometry. n=3, error bars represent SEM, *p<0.05, **p<0.01 as compared to DMSO treated controls and assessed by two-way ANOVA+Tukey-Kramer multiple comparisons post-hoc test.

FIG. 16 shows an exemplary model of AHR activity in human developmental hematopoiesis. AHR inhibition mediated by SR-1 (black arrows) enhances the differentiation of both endothelial cells (ECs) and CD34⁺ hematopoietic progenitor cells. AHR hyperactivation mediated by TCDD (gray arrows) reciprocally acts to attenuate both EC and CD34⁺ hematopoietic progenitor cells. Once CD34⁺ have been differentiated, AHR inhibition deters further differentiation into CD34⁻ terminally matured hematopoietic cells, while AHR hyperactivation supports this process. Upon production of natural killer progenitor (NKP) cells, AHR inhibition promotes conventional NK cell differentiation (cNK), while AHR hyperactivation promotes Group 3 ILC (ILC3) differentiation.

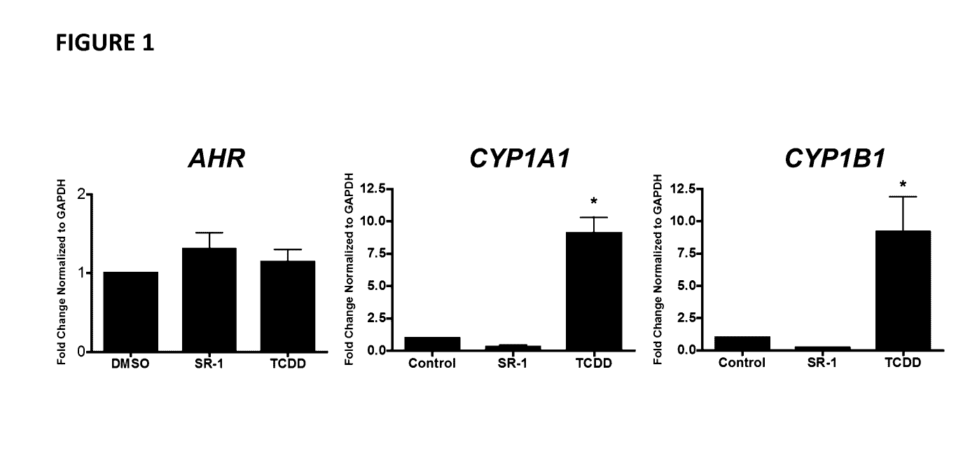

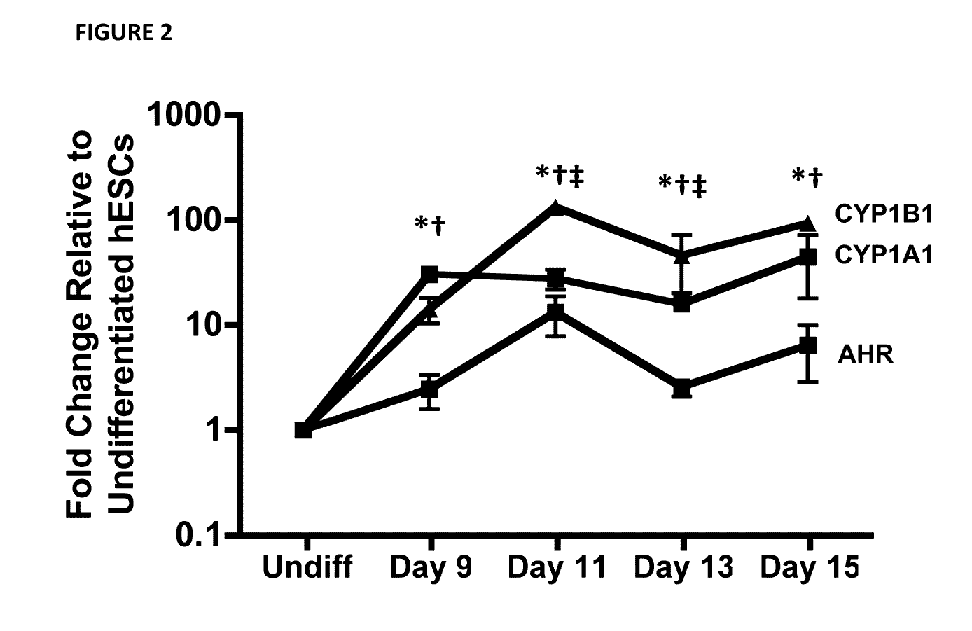

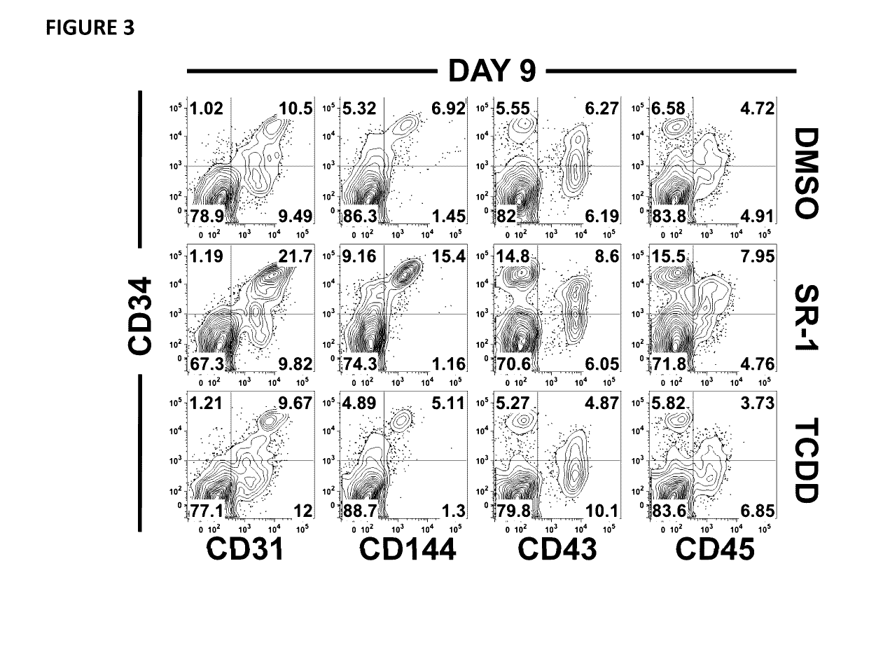

### DETAILED DESCRIPTION

This disclosure provides methods for enhancing or disrupting activity of the aryl hydrocarbon receptor (AHR), an evolutionarily conserved transcription factor, in a cell. In some embodiments, the cell is a stem cell and the regulation of activity occurs during the hematopoietic differentiation of the cell. In some aspects, the methods include antagonizing or disrupting the expression of AHR. In some aspects, the methods include hyperactivating or overexpressing AHR. In some embodiments, the stem cell is preferably a human embryonic stem cell. The antagonism, disruption, hyperactivation, and/or overexpression of AHR may be used to enhance particular types of cell differentiation from a stem cell.

In some embodiments, antagonism or disruption of AHR may be used to enhance hemato-endothelial cell development or differentiation; hematopoietic progenitor cell development or differentiation; and/or hemato-lymphoid cell development or differentiation.

In some embodiments, hyperactivation or overexpression of AHR may be used to enhance stem cell differentiation towards a hematopoietic or hemato-lymphoid cell including, for example, an innate lymphoid group 3 (ILC3) cell and/or a CD34⁻CD43⁺ cell.

Enhancing development or differentiation can include accelerating differentiation or increasing the proportion of cells in a population of cells that undergo differentiation.

This disclosure also provides a cell having disrupted or enhanced AHR activity. The cell can include an AHR mutation including, for example, deletion of at least a portion of AHR. When the cell includes an AHR mutation, the cell can be homozygous or heterozygous for the AHR mutation. The cell can include, for example, a stem cell, a hemato-endothelial cell, a hematopoietic stem/progenitor cell, or a hemato-lymphoid cell.

The aryl hydrocarbon receptor (AHR) is a member of the PAS family of environment-sensing, basic helix-loop-helix transcriptional regulators. AHR homology is evolutionarily conserved across diverse species (Schneider et al. Int. J. Mol. Sci. 15, 17852-17885 (2014)). AHR was originally characterized for its role in mediating biological responses to carcinogenic environmental agents, and it has historically been studied for its subsequent activation by environmental pollutants and as a mediator of chemical toxicity. AHR is expressed in many tissues during embryonic development and its signaling affects endogenous genes that influence development, proliferation, differentiation, and/or the innate immune response.

At the time of the invention, the role of AHR in the process of early human hematopoietic lineage commitment and the involvement of AHR signaling in the development of mesoderm-derived cells into primitive and/or definitive hematopoietic cells was unknown. This disclosure describes the effect of altering AHR expression and/or activity during early human hemato-endothelial cell, hematopoietic progenitor cell, and/or a hemato-lymphoid cell development as well as methods and techniques for manipulating the expression and/or activity of AHR. For example, as described in Examples 1 and 2, AHR expression promotes maintaining hematopoietic stem cell quiescence and hematopoietic progenitor cell differentiation, while AHR repression or disruption can promote expansion and maturation of a hemato-endothelial cell, a hematopoietic progenitor cell, and/or a hemato-lymphoid cell including, for example, a progenitor NK cell or a cNK cell.

Although Gori et al. Blood. 2012; 120(13):35-45, assessed the effect of the AHR antagonist StemReginin-1 (SR-1) in a non-human primate iPSC model of hematopoiesis and showed an increase in phenotypic CD34⁺CD45⁺ cells, that study found there were no differences in the kinetics or quantity of CD34⁺ or CD34⁺CD31⁺ cells. SR-1 treatment of non-human primate iPSCs also did not enhance the total number of colony-forming units (CFUs). Therefore, it was unexpected that, as further described herein, AHR antagonism of human cells can have dramatic effects on CD34⁺CD31⁺ and CD34⁺CD45⁺ early hemato-endothelial progenitor differentiation from human embryonic stem cells (hESCs) as well as the number of colonies generated and the proportion of cell types generated in those colonies. Without wishing to be bound by theory, it is believed that these differences between AHR ligand selectivity and AHR interaction with co-activator motifs are due to differences between non-human and primary human cells and/or to the cell culture conditions.

Also shown in Example 2, hyperactivation of AHR can suppress development of hematopoietic progenitor cells with multilineage potential and accelerate the differentiation of cells into more matured hematopoietic lineages including, for example, innate lymphoid group 3 (ILC3) cell differentiation.

In one aspect, this disclosure provides a method that includes inducing hematopoietic differentiation of a stem cell and disrupting aryl hydrocarbon receptor (AHR) activity in the cell. In some embodiments, disrupting AHR activity includes treating the cell with an AHR antagonist. In some embodiments, disrupting AHR activity includes reducing expression of AHR in the cell. In some embodiments, disrupting AHR activity includes expression of AHR having decreased activity.

In another aspect, this disclosure provides a method that includes inducing hematopoietic differentiation of a stem cell; and enhancing aryl hydrocarbon receptor (AHR) activity in the cell. In some embodiments, enhancing AHR activity includes treating the cell with an AHR agonist. In some embodiments, enhancing AHR activity includes increasing expression of AHR in the cell. In some embodiments, enhancing AHR activity includes expression of AHR having increased activity.

In some embodiments, the stem cell is an embryonic stem cell (ESC). In some embodiments, the stem cell is an induced pluripotent stem cell (iPSC). In some embodiments, the stem cell is a preferably a human stem cell. In some embodiments, the stem cell is an embryonic stem cell line, including for example, any of the cell lines listed in the NIH Human Embryonic Stem Cell Registry, available on the world wide web at grants.nih.gov/stem_cells/registry/current.htm. In some embodiments, the stem cell is an H9 human embryonic stem cell (hESC).

In some embodiments, the cell is treated with an AHR antagonist. The AHR antagonist can include, for example, StemReginin-1 (SR-1) (Boitano et al. Science. 329, 1345-1348 (2010)), CH-223191 (Kim et al. Mol. Pharmacol. 69, 1871-1878 (2006)), GNF351 (Smith et al. J. Pharmacol. Exp. Ther. 338, 318-327 (2011)), and/or 6,2,4-trimethoxyflavone (Murray et al. Pharmacol. Exp. Ther. 332, 135-144 (2010)).

In some embodiments, a preferred AHR antagonist is StemReginin-1 (SR-1). A wide variety of methods for treating a cell with an AHR antagonist can be used. In some embodiments, SR-1 can be replenished in a differentiation media every 3 days at 1 μM.

In some embodiments, a cell can be treated with an AHR antagonist before hematopoietic differentiation begins. In some embodiments, a cell may be treated with an AHR antagonist after a period of hematopoietic differentiation including, for example, up to 1 day, up to 2 days, up to 3 days, up to 4 days, up to 5 days, up to 6 days, up to 7 days, up to 8 days, up to 9 days, up to 10 days, up to 11 days, up to 12 days, up to 13 days, and/or up to 14 days of hematopoietic differentiation. In some embodiments, the cell may be treated with an AHR antagonist before and after a period of hematopoietic differentiation. In some embodiments, the cell may be treated with an AHR antagonist after the formation of an embryoid body (EB).

In some embodiments, the cell is treated with an AHR agonist. In some embodiments, the AHR agonist includes, for example, 2,3,7,8-tetrachlorodibenzo-p-dioxin (TCDD), 6-FICZ (6-Formylindolo (3,2-b)carbazole), ITE (2-(1H-Indol-3-ylcarbonyl)-4-thiazolecarboxylic acid methyl ester), meBIO ((2′Z,3′E)-6-Bromo-1-methylindirubin-3′-oxime), and/or L-Kynurenine. In some embodiments, the AHR agonist is preferably 2,3,7,8-tetrachlorodibenzo-p-dioxin (TCDD). A wide variety of methods for treating a cell with an AHR agonist can be used.

In some embodiments, a cell can be treated with an AHR agonist before hematopoietic differentiation begins. In some embodiments, a cell may be treated with an AHR agonist after a period of hematopoietic differentiation including, for example, up to 1 day, up to 2 days, up to 3 days, up to 4 days, up to 5 days, up to 6 days, up to 7 days, up to 8 days, up to 9 days, up to 10 days, up to 11 days, up to 12 days, up to 13 days, and/or up to 14 days of hematopoietic differentiation. In some embodiments, the cell may be treated with an AHR agonist before and after a period of hematopoietic differentiation. In some embodiments, the cell may be treated with an AHR agonist after the formation of an embryoid body (EB).

In some embodiments, disrupting AHR activity in the cell includes reducing expression of AHR. In some embodiments, the expression of AHR in the cell is eliminated. In some embodiments, expression is “eliminated” when the expression cannot be detected using methods known in the art including, for example, PCR. In some embodiments, expression of AHR can be reduced by treating the stem cell with siRNA. In some embodiments, a CRISPR system including, for example a CRISPR system including a dead nuclease may be used to reduce the expression of AHR. For example, the stem cell may express a catalytically inactive or dead Cas9 nuclease (dCas9) and/or a fusion of Cas9 and a transcriptional repressor. In some embodiments, the elimination of expression can be inducible. For example, expression of the dead nuclease (e.g., dCas9) and/or a fusion of the nuclease and a transcriptional repressor can be inducible.

In some embodiments, the AHR gene can include a mutation. In some embodiments, including, for example, when the method includes disrupting aryl hydrocarbon receptor (AHR) activity, the mutation can include an inactivating mutation or a deletion. In some embodiments, including, for example, when the method includes enhancing aryl hydrocarbon receptor (AHR) activity, the mutation can include an activating mutation.

In some embodiments, a mutation can include deleting at least a portion of the AHR gene. For example, at least a portion of an AHR coding region and/or an AHR promoter region can be deleted. The AHR gene includes an AHR coding region that include polynucleotides that form codons that form the AHR protein and an AHR promoter region. Deleting at least a portion of AHR may be accomplished by any method known to a skilled artisan including, for example, using a CRISPR/Cas9 system, zinc finger nucleases (ZFNs), Transcription Activator-Like Effector Nucleases (TALENs), and/or engineered meganuclease re-engineered homing endonucleases.

In some embodiments the deletion may be of a single base pair, of up to 10 base pairs, of up to 20 base pairs, of up to 50 base pairs, of up to 100 base pairs, up to 1000 base pairs, or of up to 2000 base pairs. In some embodiments, the deletion may be of the entire AHR coding region. In some embodiments, the deletion may be of the AHR coding region and AHR promoter region. In some embodiments, the deletion may be induced at a specific stage of hematopoietic differentiation. In some embodiments, the mutation includes at least one of a point mutation, a nonsense mutation (i.e. a mutation that changes a codon to a stop codon), an insertion, and/or a frameshift mutation.

In some embodiments, reducing the expression of AHR can be inducible. For example, a cell may be capable of inducing repression of the expression of an AHR coding region at a certain time point of hematopoietic differentiation. Alternatively, deleting at least a portion of the AHR gene may be induced at a certain time point of hematopoietic differentiation. In some embodiments, an inducible CRISPR/Cas9 system may be used to knock out at least a portion of the AHR gene at a certain time point of hematopoietic differentiation (see, e.g., Rathe et al. Sci. Rep. 4:6048 (2014)).

In some embodiments, enhancing AHR activity includes increasing the expression of AHR in a cell. A wide variety of methods for increasing the expression of AHR may be used including, for example, genetic overexpression, the use drugs and/or molecules that enhance activity, etc. In some embodiments, increasing the expression of AHR can be inducible. For example, a cell may be capable of inducing increased expression of an AHR coding region at a certain time point of hematopoietic differentiation.

In some embodiments, hematopoietic differentiation can include forming embryoid bodies (EB) including, for example spin EBs and/or floating EBs; stromal cell-driven differentiation; and/or 2D culture, for example, using cytokines, morphogens to induce differentiation (Tian et al., Methods Mol. Biol. 430:119-33 (2008); Tian et al. Exp. Hematol. 32(10):1000-9 (2004)). In some embodiments, hematopoietic differentiation can include subjecting cells to the culture methods described in, for example, Ng et al. Nat. Protoc. 3, 768-76 (2008); Knorr et al. Stem Cells Dev. 22, 1861-9 (2013); and/or Ferrell et al., Stem Cells 33(4), 1130-41 (2015). In some embodiments, cells may be treated with a Bovine serum albumin Polyvinyl alcohol Essential Lipids (BPEL) medium. In some embodiments, the cells may be treated with at least one of bone morphogenic protein-4 (BMP-4), vascular endothelial growth factor (VEGF), and/or stem cell factor (SCF).

In some embodiments, the methods described herein may be used to produce and/or differentiate a hematopoietic stem cell and/or a hematopoietic cell from a stem cell.

In some embodiments, the cell may be treated to induce hemato-endothelial cell differentiation (Tian et al., Methods Mol. Biol. 430:119-33 (2008); Tian et al. Exp. Hematol. 32(10):1000-9 (2004)). In some embodiments, cells may be treated with a hemato-endothelial induction medium. In some embodiments, a hemato-endothelial induction medium can include Bovine serum albumin Essential Lipids (BEL) medium. In some embodiments, a hemato-endothelial induction medium can include at least one of vascular endothelial growth factor, stem cell factor, IL-3, IL-6, and/or thrombopoietin.

In some embodiments, differentiating a cell to a hemato-endothelial cell is enhanced compared to differentiation without disrupting AHR activity. A hemato-endothelial cell can include, for example, an endothelial cell (EC), a CD34⁺CD31⁺ cell, and/or a CD34⁺CD144⁺ cell.

In some embodiments, differentiating a cell to a hematopoietic progenitor cell is enhanced compared to differentiation without disrupting AHR activity. A hematopoietic progenitor cell can include, for example, a CD34⁺CD43⁺ cell and/or a CD34⁺CD45⁺ cell.

In some embodiments, differentiating a cell to a hemato-lymphoid cell is enhanced compared to differentiation without disrupting AHR activity. A hemato-lymphoid cell can include, for example, a natural killer cell progenitor (NK progenitor) cell and/or a conventional natural killer (cNK) cell. An NK progenitor cell can include, for example, a CD94⁺CD117⁺ cell. In some embodiments, a cNK cell includes a CD56⁺CD94⁺ cell and may also be CD45⁺, KIR⁺, NKG2D⁺, NKp44⁺, NKp46⁺, and/or CD117⁻. In some embodiments, a cNK cell includes a CD56⁺CD45⁺ cell and may also be CD94⁺, KIR⁺, NKG2D⁺, NKp44⁺, NKp46⁺, and/or CD117⁻. In some embodiments, a cNK cell preferably includes a CD56⁺CD45⁺ cell or a CD94⁺CD117⁻CD56⁺CD45⁺LFA1⁻ cell. In some embodiments, a cNK cell is further defined as a cell that induces target cell lysis as measured using a chromium release cytotoxicity assay or a CD107a degranulation assay.

“Enhanced” differentiation can include, for example, a greater proportion of a population of cells undergoing differentiation to a hemato-endothelial cell, a hematopoietic progenitor cell, or a hemato-lymphoid cell and/or an accelerated differentiation to a hemato-endothelial cell, a hematopoietic progenitor cell, or a hemato-lymphoid cell. For example, in embodiments including a population of cells undergoing hematopoietic differentiation, at least 5% more, at least 10% more, at least 20% more, at least 30% more, at least 40%, at least 50%, at least 60%, at least 70%, at least 80%, at least 90%, at least 100%, at least 150% more cells can differentiate to a hemato-endothelial cell, a hematopoietic progenitor cell, or a hemato-lymphoid cell. In some embodiments, the cell can differentiate to a hemato-endothelial cell, a hematopoietic progenitor cell, or a hemato-lymphoid cell at least 5%, at least 10%, at least 20%, at least 30%, or at least 40% faster than the rate of differentiation of a cell without disrupting AHR activity.

In some embodiments, the cell may be treated to induce NK cell differentiation including, for example, progenitor NK cell differentiation and cNK cell differentiation. In some embodiments, NK cell differentiation can include subjecting cells to the culture methods and medium described in, for example, Ni et al. Methods Mol. Biol. 1029, 33-41 (2013). In some embodiments, cells may be treated with an NK cell differentiation medium. In some embodiments, an NK cell differentiation medium can include at least one of IL-15, IL-7, Flt-3 ligand, stem cell factor (SCF), and IL-3. In some embodiments, an NK cell differentiation medium can preferably include IL-15, IL-7, Flt-3 ligand, stem cell factor (SCF), and IL-3.

In some embodiments, differentiating a cell to an ILC3 cell is enhanced compared to a cell wherein hematopoietic differentiation of a stem cell has been induced and AHR activity in the cell has not been enhanced. In some embodiments, an ILC3 cell is at CD94⁻, CD117⁺, CD56⁺, and/or LFA1⁻. In some embodiments, an ILC3 cell is preferably CD94⁻, CD117⁺, CD56⁺, and LFA1⁻. In some embodiments, an ILC3 cell expresses at least one of RORc, IL1-R1, and IL-22. In some embodiments, an ILC3 cell expresses RORc, IL1-R1, and IL-22.

“Enhanced” differentiation can include, for example, a greater proportion of a population of cells undergoing differentiation to an ILC3 cell and/or an accelerated differentiation to a ILC3 cell. For example, in embodiments including a population of cells undergoing hematopoietic differentiation, at least 5% more, at least 10% more, at least 20% more, at least 30% more, or at least 40% more cells in a population of stem cells can differentiate to an ILC3 cell. In some embodiments, the cells can differentiate to a ILC3 cell at least 5%, at least 10%, at least 20%, at least 30%, or at least 40% faster than the rate of differentiation without enhancement of AHR activity.

In some embodiments, the cell may be treated to induce innate lymphoid group 3 (ILC3) cell differentiation. In some embodiments, cells may be treated with an NK cell differentiation medium. In some embodiments, an NK cell differentiation medium can include at least one of IL-15, IL-7, Flt-3 ligand, stem cell factor (SCF), and IL-3. In some embodiments, an NK cell differentiation medium can preferably include IL-15, IL-7, Flt-3 ligand, stem cell factor (SCF), and IL-3.

In some embodiments, including when the method includes enhancing AHR activity in the cell, the methods described herein may be used to enhance innate lymphoid cell group 3 (ILC3) cell differentiation or to enhance differentiation of a hematopoietic cell, e.g., a CD34⁻CD43⁺ cell. In some embodiments, an ILC3 cell expresses at least one of RORc, IL1-R1, and IL-22, and/or expresses lower levels of at least one of GATA3 and TBX21/TBET compared to a stem cell. In some embodiments, an ILC3 cell expresses RORc, IL1-R1, and IL-22.

In some embodiments, the method can include selecting, e.g., by flow cytometry, a cell or a cell population. For example, the method could include selecting for a hemato-endothelial cell, a hematopoietic progenitor cell, and/or a hemato-lymphoid cell. A hemato-endothelial cell can include, for example, an endothelial cell (EC), a CD34⁺CD31⁺ cell, and/or a CD34⁺CD144⁺ cell. A hematopoietic progenitor cell can include, for example, a CD34⁺CD43⁺ cell, and/or a CD34⁺CD45⁺ cell. A hemato-lymphoid cell can include, for example, a natural killer cell progenitor (NK progenitor) cell, a conventional natural killer (cNK) cell, and/or an ILC3 cell. In some embodiments, the method can include selecting for a cell that expresses a cell marker or gene. This disclosure also describes a cell derived from the methods described herein. In some embodiments, the cell includes a hemato-endothelial cell; a hematopoietic progenitor cell; a hemato-lymphoid cell; an endothelial cell (EC); a hematopoietic cell; an NK progenitor cell; a cNK cell; an innate lymphoid cell group 3 (ILC3) cell; and/or a differentiated hematopoietic cell; e.g., a CD34⁻CD43⁺ cell. In some embodiments, the cell includes a mutation in AHR.

This disclosure also describes a composition including a cell derived from the methods described herein. In some embodiments, the composition further includes a pharmaceutically acceptable carrier. The pharmaceutically acceptable carrier can include, for example, an excipient, a diluent, a solvent, an accessory ingredient, a stabilizer, a protein carrier, or a biological compound. Non-limiting examples of a protein carrier includes keyhole limpet hemocyanin (KLH), bovine serum albumin (BSA), ovalbumin, or the like. Non-limiting examples of a biological compound which can serve as a carrier include a glycosaminoglycan, a proteoglycan, and albumin. The carrier can be a synthetic compound, such as dimethyl sulfoxide or a synthetic polymer, such as a polyalkyleneglycol. Ovalbumin, human serum albumin, other proteins, polyethylene glycol, or the like can be employed as the carrier. In a preferred embodiment, the pharmaceutically acceptable carrier includes at least one compound that is not naturally occurring or a product of nature.

In some embodiments, the cell derived from the methods described herein can be transplanted and/or engrafted using methods known to a skilled artisan. See, e.g., Tian et al. Stem Cells 27, 2675-85 (2009); Tian et al. Stem Cells, 24, 1370-1380 (2006); Hexum et al., “In Vivo Evaluation of Putative Hematopoietic Stem Cells Derived from Pluripotent Stem Cells,” in Human Pluripotent Stem Cells, Schwartz et al. (Eds.) Springer Science, 2011.

In a further aspect, this disclosure provides a cell having disrupted or enhanced AHR activity. The cell can include an AHR mutation including, for example, deletion of at least a portion of AHR. When the cell includes a mutation, the cell can be homozygous or heterozygous for the AHR mutation. The cell can include, for example, a stem cell; a hematopoietic progenitor cell; an EC; a hematopoietic cell; an NK progenitor cell; a cNK cell; an ILC3 cell; and/or a differentiated hematopoietic cell, e.g., a CD34⁻CD43⁺ cell. In some embodiments, the cell is preferably a human cell.

In some embodiments, the cell is a stem cell including, for example, an embryonic stem cell. In some embodiments, the cell is a cell derived from a stem cell including, for example, a hemato-endothelial cell, a hematopoietic progenitor cell, and a hemato-lymphoid cell. In some embodiments, the hemato-endothelial cell, the hematopoietic progenitor cell, and/or the hemato-lymphoid cell can be derived from a stem cell that is homozygous or heterozygous for an AHR mutation.

A hemato-endothelial cell can include, for example, an endothelial cell (EC), a CD34⁺CD31⁺ cell, and/or a CD34⁺CD144⁺ cell. A hematopoietic progenitor cell can include, for example, a CD34⁺CD43⁺ cell, and/or a CD34⁺CD45⁺ cell. A hemato-lymphoid cell can include, for example, a natural killer cell progenitor (NK progenitor) cell, a conventional natural killer (cNK) cell, and/or an ILC3 cell.

An NK progenitor cell can include, for example, a CD94⁺CD117⁺ cell. In some embodiments, a cNK cell includes a CD56⁺CD94⁺ cell and may also be CD45⁺, KIR⁺, NKG2D⁺, NKp44⁺, NKp46⁺, and/or CD117⁻. In some embodiments, a cNK cell includes a CD56⁺CD45⁺ cell. and also may be CD94⁺, KIR⁺, NKG2D⁺, NKp44⁺, NKp46⁺, and/or CD117⁻. In some embodiments, a cNK cell preferably includes a CD56⁺CD45⁺ cell or a CD94⁺CD117⁻CD56⁺CD45⁺LFA1⁻ cell. In some embodiments, an ILC3 cell is at CD94⁻, CD117⁺, CD56⁺, and/or LFA1⁻. In some embodiments, a cNK cell is further defined as a cell that induces target cell lysis as measured using a chromium release cytotoxicity assay or a CD107a degranulation assay.

In some embodiments, an ILC3 cell is preferably CD94⁻, CD117⁺, CD56⁺, and LFA1⁻. In some embodiments, an ILC3 cell expresses at least one of RORc, IL1-R1, and IL-22. In some embodiments, an ILC3 cell expresses RORc, IL1-R1, and IL-22.

In some embodiments, the mutation can include a mutation in the coding region of AHR. In some embodiments, the mutation can include a mutation in the promoter region of AHR. In some embodiments, the mutation includes a deletion of at least a portion of AHR. In some embodiments the deletion may be of a single base pair, of up to 10 base pairs, of up to 20 base pairs, of up to 50 base pairs, of up to 100 base pairs, up to 1000 base pairs, or of up to 2000 base pairs. In some embodiments, the deletion may be of the entire AHR coding region. In some embodiments, the deletion may be of the AHR coding region and AHR promoter region. In some embodiments, the deletion may be induced at a specific stage of hematopoietic differentiation. In some embodiments, the mutation includes at least one of a point mutation, a nonsense mutation (i.e. a mutation that changes a codon to a stop codon), an insertion, and/or a frameshift mutation.

In some embodiments, the mutation is an activating mutation, that is, a mutation that increases the activity of AHR.

This disclosure also describes a composition including the cell. In some embodiments, the composition further includes a pharmaceutically acceptable carrier. The pharmaceutically acceptable carrier can include, for example, an excipient, a diluent, a solvent, an accessory ingredient, a stabilizer, a protein carrier, or a biological compound. Non-limiting examples of a protein carrier includes keyhole limpet hemocyanin (KLH), bovine serum albumin (B S A), ovalbumin, or the like. Non-limiting examples of a biological compound which can serve as a carrier include a glycosaminoglycan, a proteoglycan, and albumin. The carrier can be a synthetic compound, such as dimethyl sulfoxide or a synthetic polymer, such as a polyalkyleneglycol. Ovalbumin, human serum albumin, other proteins, polyethylene glycol, or the like can be employed as the carrier. In a preferred embodiment, the pharmaceutically acceptable carrier includes at least one compound that is not naturally occurring or a product of nature.

In some embodiments, the cell can be transplanted and/or engrafted using methods known to a skilled artisan. See, e.g., Tian et al. Stem Cells 27, 2675-85 (2009); Tian et al. Stem Cells, 24, 1370-1380 (2006); Hexum et al., “In Vivo Evaluation of Putative Hematopoietic Stem Cells Derived from Pluripotent Stem Cells,” in Human Pluripotent Stem Cells, Schwartz et al. (Eds.) Springer Science, 2011.

### ILLUSTRATIVE EMBODIMENTS

### Embodiment 1

A method comprising
inducing hematopoietic differentiation of a stem cell; and
disrupting aryl hydrocarbon receptor (AHR) activity in the cell.

### Embodiment 2

The method of Embodiment 1, wherein disrupting AHR activity comprises treating the cell with an aryl hydrocarbon receptor (AHR) antagonist.

### Embodiment 3

The method of Embodiment 2, wherein the AHR antagonist comprises StemReginin-1 (SR-1), GNF351, or 6,2,4-trimethoxyflavone.

### Embodiment 4

The method of Embodiment 1, wherein disrupting AHR activity comprises reducing expression of AHR in the cell.

### Embodiment 5

The method of Embodiment 4, wherein the method comprises eliminating expression of AHR.

### Embodiment 6

The method of either of Embodiments 4 or 5, wherein at least a portion of an AHR coding region or an AHR promoter region or both is mutated.

### Embodiment 7

The method of any of Embodiments 4 to 6, wherein the reduction of expression of AHR is inducible.

### Embodiment 8

The method of any of Embodiments 1 to 7, wherein the stem cell is an embryonic stem cell or an induced pluripotent stem cell.

### Embodiment 9

The method of Embodiment 8, wherein the embryonic stem cell is an H9 human embryonic stem cell.

### Embodiment 10

The method of any of Embodiments 1 to 9, wherein differentiation of the cell to a at least one of a hemato-endothelial cell, a hematopoietic progenitor cell, and a hemato-lymphoid cell is enhanced compared to differentiation without disruption of AHR activity.

### Embodiment 11

The method of any of Embodiments 1 to 10, wherein the method comprises treating the cell to enhance at least one of hemato-endothelial cell differentiation, hematopoietic progenitor cell differentiation, and hemato-lymphoid cell differentiation.

### Embodiment 12

The method of either of Embodiments 10 or 11, wherein the hemato-endothelial cell comprises at least one of an endothelial cell (EC), a CD34⁺CD31⁺ cell, and a CD34⁺CD144⁺cell.

### Embodiment 13

The method of any of Embodiments 10 to 12, wherein the hematopoietic progenitor cell comprises at least one of a CD34⁺CD43⁺ cell and a CD34⁺CD45⁺ cell.

### Embodiment 14

The method of any of Embodiments 10 to 13, wherein the hemato-lymphoid cell comprises at least one of a natural killer cell progenitor (NK progenitor) cell and a conventional natural killer (cNK) cell.

### Embodiment 15

The method of any of Embodiments 1 to 14, wherein the method comprises treating the cell with a hemato-endothelial induction medium comprising at least one of vascular endothelial growth factor, stem cell factor, IL-3, IL-6, and thrombopoietin.

### Embodiment 16

The method of any of Embodiments 1 to 15, wherein the method comprises treating the cell to enhance at least one of NK progenitor cell differentiation and cNK cell differentiation.

### Embodiment 17

The method of any of Embodiments 1 to 16, wherein the method comprises treating the cell with NK cell differentiation medium comprising at least one of IL-15, IL-7, Flt-3 ligand, stem cell factor, and IL-3.

### Embodiment 18

The method of any of Embodiments 1 to 17, wherein the method further comprises enhancing at least one of NK progenitor cell differentiation and cNK cell differentiation.

### Embodiment 19

The method of any of Embodiments 1 to 18, wherein the stem cell is a human cell.

### Embodiment 20

A cell derived from the methods according to any one of Embodiments 1 to 19.

### Embodiment 21

The cell of Embodiment 20 comprising a hemato-endothelial cell, a hematopoietic progenitor cell, or a hemato-lymphoid cell.

### Embodiment 22

The cell of either of Embodiments 20 or 21 wherein the cell comprises a mutation in AHR.

### Embodiment 23

A cell being homozygous or heterozygous for an aryl hydrocarbon receptor (AHR) mutation.

### Embodiment 24

The cell of Embodiment 23, wherein the mutation comprises a mutation in the coding region of AHR.

### Embodiment 25

The cell of either of Embodiments 23 or 24, wherein the cell is a stem cell.

### Embodiment 26

The cell of Embodiment 23, wherein the stem cell is an embryonic stem cell.

### Embodiment 27

The cell of either of Embodiments 23 or 24, wherein the cell is a hemato-endothelial cell, a hematopoietic progenitor cell, or a hemato-lymphoid cell.

### Embodiment 28

The cell of either of Embodiments 23 or 24, wherein the cell is a natural killer cell progenitor (NK progenitor) cell, a conventional natural killer (cNK) cell, an endothelial cell (EC), or an innate lymphoid group 3 (ILC3) cell.

### Embodiment 29

The cell of any of Embodiments 23 to 28, wherein the cell is a human cell.

### Embodiment 30

A method comprising inducing hematopoietic differentiation of a stem cell; and enhancing aryl hydrocarbon receptor (AHR) activity in the cell.

### Embodiment 31

The method of Embodiment 30, wherein enhancing AHR activity comprises treating the cell with an AHR agonist.

### Embodiment 32

The method of Embodiment 31, wherein the AHR agonist comprises tetrachlorodibenzo-p-dioxin (TCDD), 2,3,7,8-tetrachlorodibenzo-p-dioxin (TCDD), 6-FICZ (6-Formylindolo (3,2-b)carbazole), ITE (2-(1H-Indol-3-ylcarbonyl)-4-thiazolecarboxylic acid methyl ester), meBIO ((2′Z,3′E)-6-Bromo-1-methylindirubin-3′-oxime), or L-Kynurenine.

### Embodiment 33

The method of Embodiment 30, wherein enhancing AHR activity comprises increasing expression of aryl hydrocarbon receptor (AHR) in a stem cell.

### Embodiment 34

The method either of Embodiments 30 or 33, wherein at least a portion of an AHR coding region or an AHR promoter region or both is mutated.

### Embodiment 35

The method of any of Embodiments 30, 33, or 34, wherein the enhancement of AHR activity is inducible.

### Embodiment 36

The method of any of Embodiments 30 to 35, wherein the stem cell is an embryonic stem cell or an induced pluripotent stem cell.

### Embodiment 37

The method of Embodiment 36, wherein the embryonic stem cell is an H9 human embryonic stem cell.

### Embodiment 38

The method of any of Embodiments 30 to 37, wherein differentiation of the cell to an innate lymphoid cell group 3 (ILC3) cell is enhanced compared to differentiation without enhancement of AHR activity.

### Embodiment 39

The method of any of Embodiments 30 to 38, wherein the method further comprises treating the cell to enhance hemato-endothelial cell differentiation.

### Embodiment 40

The method of Embodiment 39, wherein the method comprises treating the cell with hemato-endothelial induction medium.

### Embodiment 41

The method of Embodiment 40, wherein the hemato-endothelial induction medium comprises at least one of vascular endothelial growth factor, stem cell factor, IL-3, IL-6, and thrombopoietin.

### Embodiment 42

The method of any of Embodiments 30 to 41, wherein the method further comprises treating the cell to enhance differentiation of an innate lymphoid cell group 3 (ILC3) cell or a CD34⁻, CD43⁺ hematopoietic cell.

### Embodiment 43

The method of either of Embodiment 38 to 42, wherein the ILC3 cell is at least one of CD94⁻, CD117⁺, CD56⁺, and LFA1⁻.

### Embodiment 44

The method of any of Embodiments 38 to 43, wherein the ILC3 cell
expresses at least one of RORc, IL1-R1, and IL-22, and/or
expresses lower levels of at least one of GATA3 and TBX21/TBET compared to the stem cell.

### Embodiment 45

The method of any of Embodiments 30 to 44, wherein the method comprises treating the cell with NK cell differentiation medium.

### Embodiment 46

The method of Embodiment 45, wherein the NK cell differentiation medium comprises at least one of IL-15, IL-7, Flt-3 ligand, stem cell factor, and IL-3.

### Embodiment 47

The method of any of Embodiments 30 to 46, wherein the method comprises enhancing differentiation of CD34⁻CD43⁺ hematopoietic cells, differentiation of innate lymphoid cell group 3 (ILC3) cells, or both.

### Embodiment 48

The method of any of Embodiments 30 to 47, wherein the stem cell is a human cell.

### Embodiment 49

A cell derived from the methods according to any one of Embodiments 30 to 48.

### Embodiment 50

The cell of Embodiment 49 comprising an innate lymphoid cell group 3 (ILC3) cell.

### Embodiment 51

The cell of either of Embodiments 49 or 50 wherein the cell comprises a mutation in AHR.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Materials and Methods

### Human Embryonic Stem (ES) Cell Culture and Hemato-Endothelial Differentiation

H9 human embryonic stem cells (H9 hESCs; University of Wisconsin, Madison, Wis.) were adapted to passage as single cells using TrypLE (Invitrogen, Carlsbad, Calif.) as previously described (Ng et al. Nat. Protoc. 3, 768-76 (2008)). H9 hESCs were maintained on irradiated mouse embryonic fibroblasts in ES cell growth medium including Dulbecco's modified Eagle's medium (DMEM)/Ham's F-12 medium (F12) (Invitrogen) supplemented with 15% Knockout Serum Replacer (Invitrogen), 1 mM L-glutamine (Invitrogen), 0.1 mM β-mercaptoethanol (Sigma Aldrich, St. Louis, Mo.), 1% minimum essential medium nonessential amino acids (Invitrogen), 4 ng/mL basic fibroblast growth factor (R&D Systems Inc., Minneapolis, Minn.), and 1% penicillin-streptomycin (Invitrogen) incubated at 37° C. in a humidified atmosphere containing 5% CO₂. Single-cell adapted hESCs were subjected to hemato-endothelial differentiation as spin embryoid bodies (“spin-EBs”), also previously described (Ng et al. Nat. Protoc. 3, 768-76 (2008); Knorr et al. Stem Cells Dev. 22, 1861-9 (2013); Ferrell et al., Stem Cells 33(4), 1130-41 (2015)). In brief, TrypLE-adapted hESCs were harvested are resuspended in bovine serum albumin polyvinyl alcohol essential lipids (BPEL) media (Ferrell et al. Stem Cells 33(4):1130-41 (2015)) supplemented with 20 ng/mL bone morphogenic protein-4 (R&D Systems), 20 ng/mL vascular endothelial growth factor (R&D Systems), and 40 ng/mL stem cell factor (R&D Systems) (hemato-endothelial induction media or Stage I mesodermal induction media). Cells were plated in a low-attachment 96-well plate (Thermo Scientific, Waltham, Mass.) at 3,000 cells per well and centrifuged at 1500 rpm for 5 minutes to create three-dimensionalized embryoid body aggregates. Following 6 days of incubation, EBs were transferred with a multichannel micropipette to 0.1% gelatinized 24-well plates (8 EBs/well) with BEL media (BPEL media minus polyvinyl alcohol) supplemented with 40 ng/mL vascular endothelial growth factor, 40 ng/mL stem cell factor, 30 ng/mL Interleukin-3 (Peprotech, Rocky Hill, N.J.), 30 ng/mL Interleukin-6 (Peprotech), and 30 ng/mL thrombopoietin (Peprotech) (hESC growth media or Stage II media) to induce hemato-endothelial-specific differentiation. Depending on the treatment group, Stage II media was also supplemented with DMSO, 1 μM StemReginin-1 (SR-1) (Cellagen Technology, San Diego, Calif.) in DMSO or 10 nM 2,3,7,8-tetrachlorodibenzo-p-dioxin (TCDD) (Sigma Aldrich) in DMSO. EBs were cultured until the indicated time points and one-half media changes with fresh Stage II media and drugs were performed every three days. For EB harvesting, the media containing the non-adherent differentiated hematopoietic cells was set aside while the adherent cells were treated with 0.05% trypsin (Invitrogen) containing 2% chicken serum (Sigma Aldrich) for 8-10 minutes in a 37° C. water bath. Both non-adherent and adherent fractions were combined for subsequent analyses.

Natural Killer (NK) Cell Differentiation from hESCs

H9 hESCs were differentiated into NK cells as previously described (Ni et al. Embryonic Stem Cell Immunobiol. Methods Protoc. Methods Mol. Biol. 1029, 33-41 (2013)). In brief, H9 hESCs were aggregated as spin-EBs and differentiated in Stage I mesodermal induction media for 11 days, with one-half media change with fresh cytokines on Day 6. On Day 11, spin-EBs were transferred with a multichannel pipette to 0.1% gelatinized 24-wells plates with NK-differentiation media (NKDM) consisting of DMEM-Glutamax (Invitrogen), F12/Ham's Media-Glutamax (Invitrogen), 15% Human AB Serum (Access Biologicals, Vista, Calif.), 1% L-glutamine (Invitrogen), 1% penicillin-streptomycin (Invitrogen), 25 μM β-mercaptoethanol (Sigma Aldrich), 50 μM ethanolamine, 20 μg/mL ascorbic acid (Sigma), and 5 ng/mL sodium selenite. For 1 week, NKDM was supplemented with 10 ng/mL Interleukin-15 (Peprotech), 20 ng/mL Interleukin-7 (Peprotech), 10 ng/mL Flt3-ligand (Peprotech), 20 ng/mL stem cell factor (R&D Systems), and 5 ng/mL interleukin-3 (Peprotech). Depending on the treatment group, NKDM was also supplemented with DMSO, 1 μM StemReginin-1 (SR-1) (Cellagen Technology) or 10 nM 2,3,7,8-tetrachlorodibenzo-p-dioxin (TCDD) (Sigma Aldrich). Each subsequent week, half-media exchanges were performed with fresh NKDM and drugs except Interleukin-3 was removed following one week of culture. Cells were maintained until the indicated time points in a 37° C. in a humidified atmosphere containing 5% CO₂.

### Hematopoietic Colony Forming Unit (CFU) Assay

Non-adherent, differentiated hematopoietic progenitor cells were harvested following 6 days growth as spin-EBs in Stage I conditions, followed by an additional 6 days growth in Stage II conditions. Cells were then washed with DPBS, filtered through an 80 μm cell strainer, and resuspended in Iscove's Modified Dulbecco's Medium (IMDM) (Invitrogen) containing 2% fetal bovine serum. 50,000 cells were resuspended in 2 mL of H4436 Methocult media (Stem Cell Technologies, Vancouver, Canada) and plated in a 35 mm×10 mm tissue culture dish (Greiner Bio-one, Monroe, N.C.). Plates were incubated in a 37° C. in a humidified atmosphere containing 5% CO₂ for 14 days and were subsequently scored.

### Flow Cytometry

Harvested cells were incubated with PE-Cy7 CD34 (BD Biosciences, San Jose, Calif.) in conjunction with APC CD31, APC CD41a, APC CD43, or APC CD45 for 30 minutes at 4° C. in FACS buffer (DPBS containing 2% fetal bovine serum and 0.1% sodium azide). Dead cells were excluded using Sytox Blue dye (Invitrogen) prior to analysis. Cells were analyzed using an LSRII flow cytometer (BD Biosciences) and data were analyzed using FlowJo software (TreeStart, Ashland, Oreg.).

### RNA Isolation and Quantitative Real-Time Polymerase Chain Reaction

Total RNA was harvested using the QiaShredder and RNAEasy Isolation Kits (Qiagen, Germantown, Md.) per manufacturer protocol. RNA was eluted into RNA-free water and quantified for concentration and purity using a Nanodrop spectrophotometer. 1 μg cDNA was generated using a SensiFAST cDNA Synthesis Kit (Bioline USA Inc., Taunton, Mass.). For quantitative PCR assays, 50 ng cDNA was amplified with SYBR Green qPCR mix (Applied Biosystems) and gene specific primers; AHR: F 5′-TCGGCCACGGAGTTTCTTC-3′ (SEQ ID NO:1); R 5-GGTCAGCATGTGCCCAATCA-3′ (SEQ ID NO:2); CYP1A1: F 5′-ACATGCTGACCCTGGGAAAG-3′ (SEQ ID NO:3); R 5′-GGTGTGGAGCCAATTCGGA-3′ (SEQ ID NO:4); CYP1B1: F 5′-TGAGTGCCGTGTGTTTCGG-3′ (SEQ ID NO:5); R 5-GTTGCTGAAGTTGCGGTTGAG-3′(SEQ ID NO:6); GAPDH: F 5′-CCACTCCTCCACCTTTGAC-3′ (SEQ ID NO:7); R 5′-ACCCTGTTGCTGTAGCCA-3′ (SEQ ID NO:8). PCR reactions were executed on an e² RealPlex Thermocycler (Eppendorf, Hauppauge, N.Y.). Triplicated Cₜ values for each sample were then averaged and normalized to GAPDH. Fold change was calculated using the 2^{ΔΔCt} method.

### Results

This Example describes evaluation of whether inhibition of AHR-mediated cell signaling could promote early human hematopoietic cell development. To model human hematopoiesis, a xenogeneic-free and chemically defined in vitro method was employed to differentiate human embryonic stem cells (hESCs) into endothelial and hematopoietic cells. qRT-PCR analysis demonstrated a significant increase in AHR (13.36±5.52 fold change, p<0.05, n=3) by Day 11 of differentiation relative to undifferentiated hESCs. CYP1A1 and CYP1B1, two downstream targets of AHR-mediated signaling, were similarly upregulated on Day 11 (27.90±6.17 fold change, p<0.05, n=3; 134.28±10.06 fold change, n=3, respectively). Increase in AHR expression mirrored the onset of early hematopoietic progenitor cell differentiation; CD34⁺CD43⁺ and CD34⁺CD41a⁺ cells were markedly increased by Day 12 of hematopoietic differentiation (compared to Day 9), as assessed by flow cytometry (18.9%±3.22, p<0.01, n=7; 8.23±2.00, p<0.05, n=7, respectively).

The relative activity of AHR signaling was modified by differentiating hESCs in the presence of 2,3,7,8-tetrachlorodibenzo-p-dioxin (TCDD), a prototypical AHR agonist, or StemReginin-1 (SR-1), an AHR antagonist, and assessed the effects on hematopoietic progenitor cell production. Interestingly, a significant increase in the appearance of both CD34⁺CD31⁺ hemato-endothelial cells in SR-1-treated hESCs was observed relative to DMSO-treated controls (17.63%±1.25, p<0.05, n=3 vs. 11.21±0.63, p<0.05, n=3) at Day 9. By Day 12, an approximately two-fold expansion of CD34⁺CD45⁺ hematopoietic progenitor cells in SR-1-treated hESCs was found relative to DMSO-treated controls (16.35%±4.05, p<0.05, n=3 vs. 7.53±0.19, p<0.05, n=3). Treatment with TCDD reciprocally attenuated the development of CD34⁺CD45⁺ progenitor cells at Day 15 relative to DMSO-treated controls (3.99%±0.80 vs. 11.79%±1.41, p<0.05, n=3) and resulted in an expansion of differentiated hematopoietic cells (CD34⁻CD43⁺: 84.5%±2.78 vs. 70.9±1.58, p<0.05, n=3; CD34⁻CD45⁺: 81.75%±1.75 vs. 71.95±2.35, p<0.05, n=3).

The functionality of the hematopoietic progenitor cells in each group was confirmed by harvesting non-adherent cells at Day 12 and performing standard colony-forming assays. SR-1-treated cells yielded a 4-fold increase in the total number of colonies generated relative to DMSO-treated control cells along with an increased proportion of CFU-M and CFU-GM.

Whether AHR antagonism could be used to promote NK cell differentiation from hESCs was also evaluated. Using previously optimized and defined NK cell differentiation conditions, SR-1 treatment was found to cause an increase in CD56⁺CD45⁺ NK cells relative to DMSO-treated controls (26.4% vs. 19.7%, n=2) whereas TCDD treatment caused a decrease (6.7%, n=2).

These results demonstrate 1) AHR antagonism promotes early human hemato-endothelial development from hESCs and may be used as a potential molecular target to enhance hematopoietic cell production from human pluripotent stem cells for research and clinical applications; and 2) AHR antagonism increases production of NK cells (CD45⁺CD56⁺ cells) from human pluripotent stem cells.

### Example 2

### Materials and Methods

Hemato-Endothelial Differentiation of hESCs

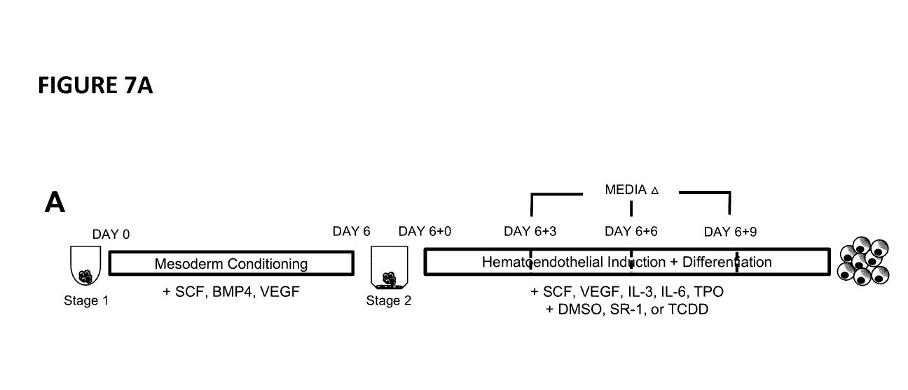

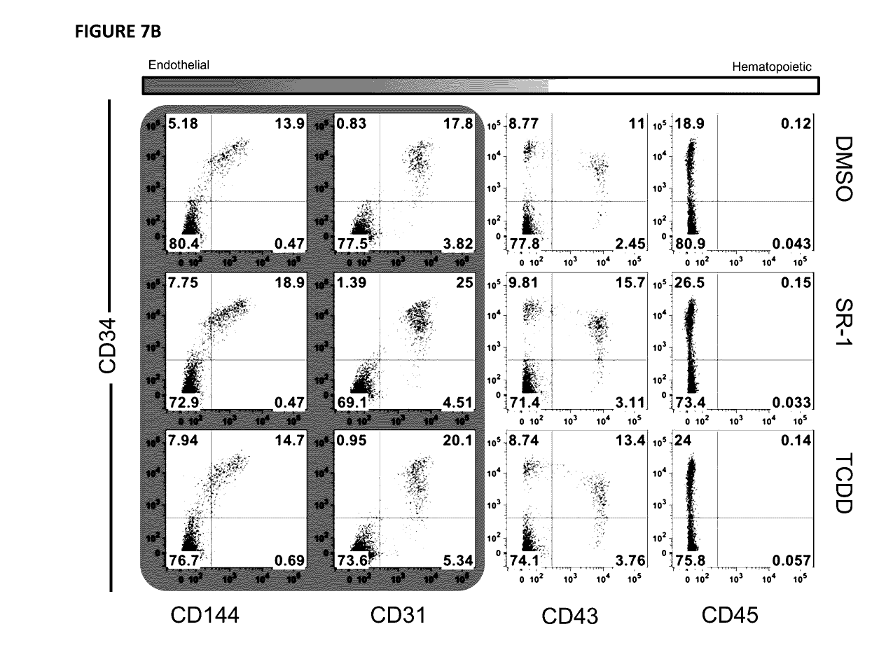

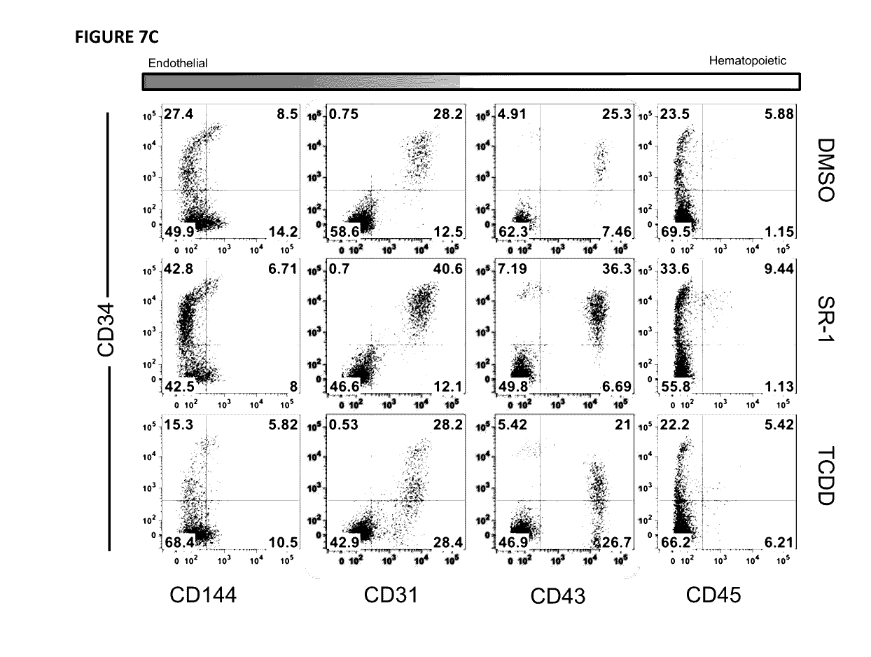

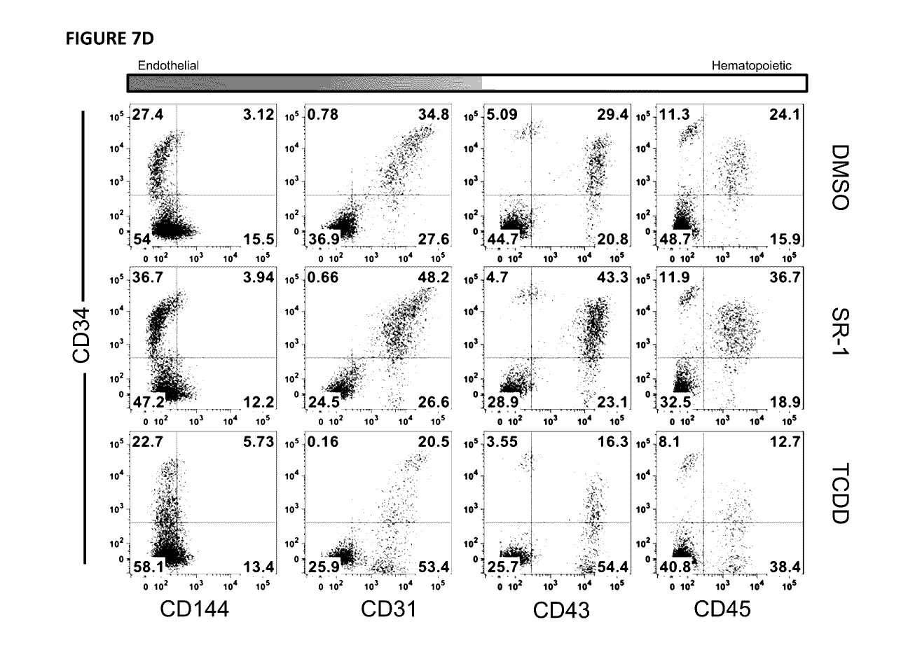

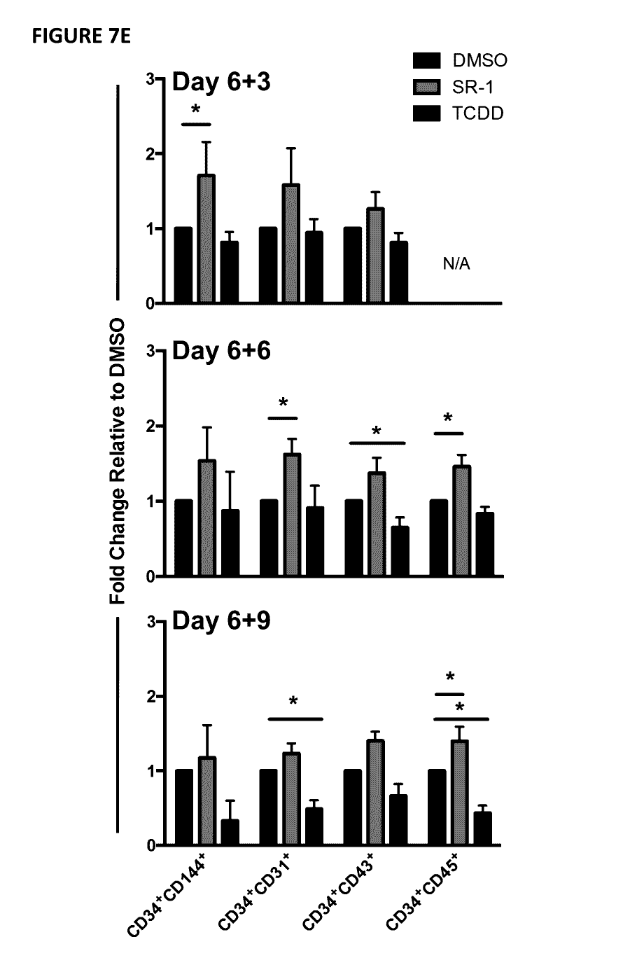

Single-cell adapted hESCs (H9) were maintained on irradiated mouse embryonic fibroblasts (MEF) in ES growth media, as previously described in Ferrell et al. Stem Cells. 2015; 33(4):1130-41. hESCs were allowed to differentiate as spin-embryoid bodies (EBs) as previously described in Ferrell et al. and Ng et al. Nat. Protoc. 2008; 3(5):768-776 (FIG. 7A). In brief, hESCs were plated at 3,000 cells/100 μL in a round-bottom 96-well plate using serum-free BPEL media supplemented with 20 ng/mL BMP4, 40 ng/mL SCF, and 20 ng/mL VEGF (all R&D Systems, Minneapolis, Minn.). Cells were centrifuged to form embryoid bodies (defined as Day 0) and were incubated for 6 additional days (defined as Day 6) to promote mesoderm induction. To differentiate early endothelial and hematopoietic progenitor cells, Day 6 EBs were transferred to pre-gelatinized 24-well plates (approx. 8-16 EBs/well) with BEL media supplemented with 40 ng/mL SCF, 40 ng/mL VEGF, 30 mg/mL thrombopoietin (all R&D Systems, Minneapolis, Minn.), 30 ng/mL IL-3, and 30 ng/mL IL-6 (both PeproTech, Rocky Hill, N.J.). To modulate AHR activity, EBs were treated at Day 6+0 with DMSO, 1 μM SR-1 (Cellagen Technologies, San Diego, Calif.), or 10 nM 2,3,7,8-tetrachlorodibenzo-p-dioxin (TCDD) (Sigma-Aldrich, St. Louis, Mo.). Media was exchanged every 3 days with small molecule and cytokine supplementation. At indicated time points, non-adherent cell fractions were collected and saved while the remaining adherent fractions were treated with 0.05% trypsin containing 2% chicken serum. Adherent cells were combined with the non-adherent fractions for analysis, unless otherwise stated.

Innate Lymphoid Cell (cNK Cell and ILC3) Differentiation from Spin-EBs

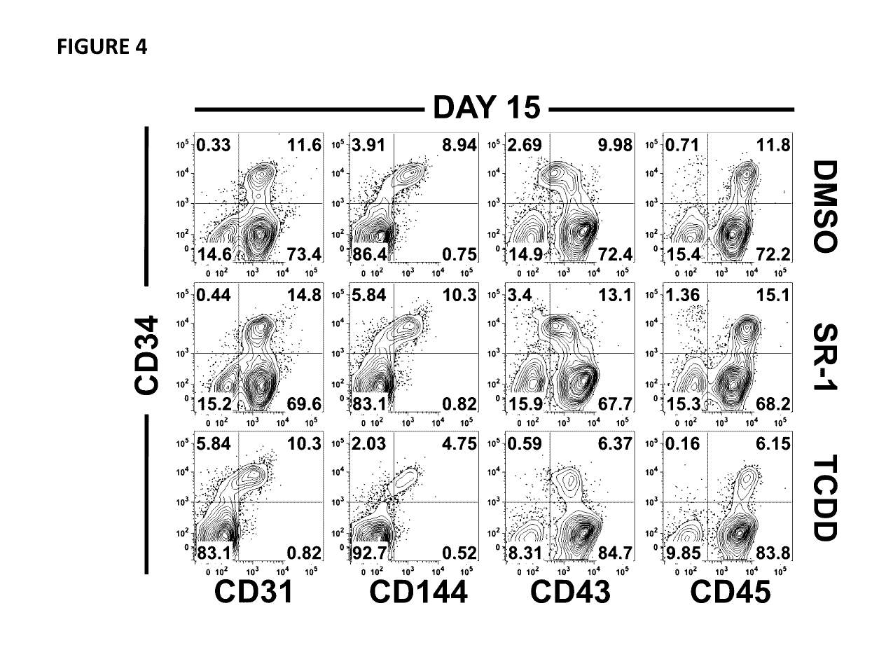

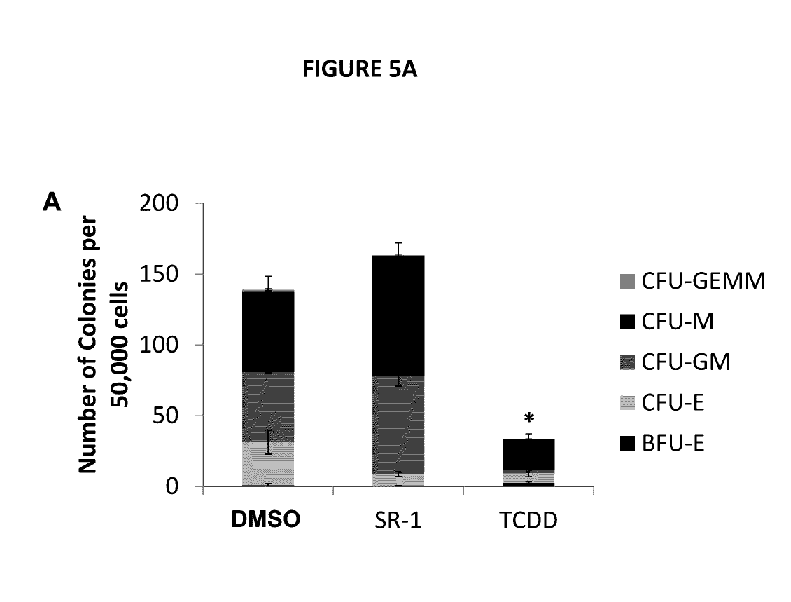

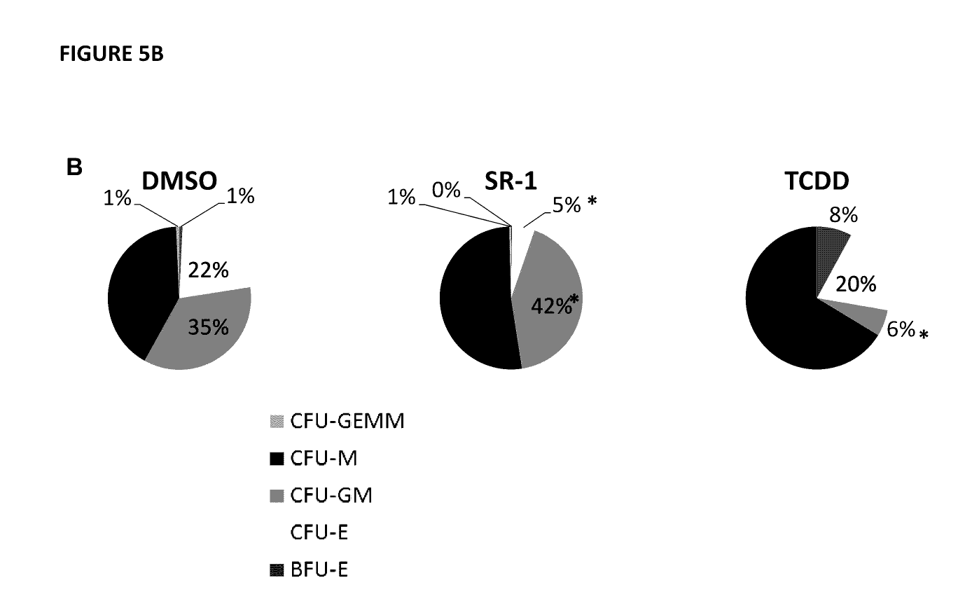

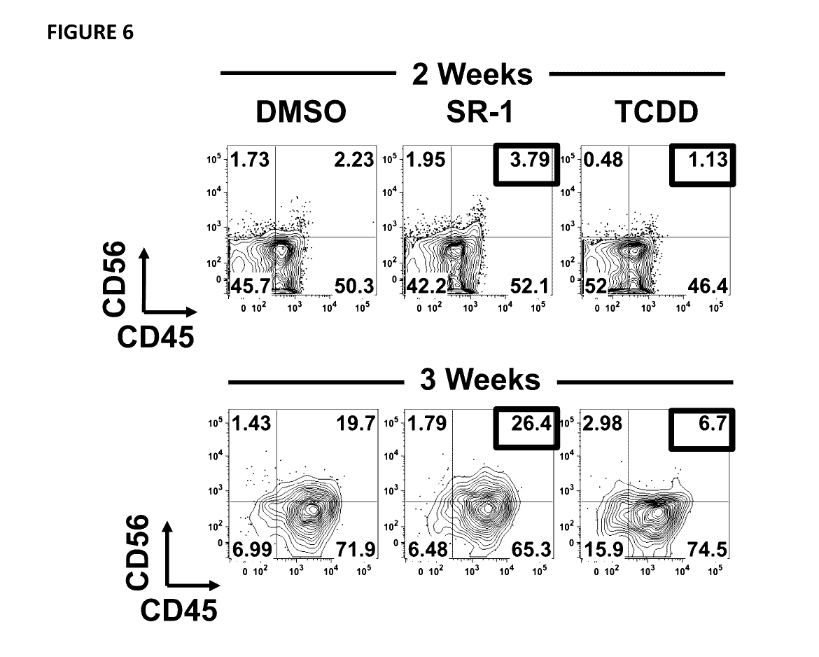

Spin-EBs were generated as described above (see also Knorr et al. Stem Cells Dev. 2013; 22(13):1861-9; Ni et al. Embryonic Stem Cell Immunobiol. Methods Protoc. Methods Mol. Biol. 2013; 1029:33-41; Hermanson et al. Stem Cells. 2016; 34(1):93-101). Following 11 days of mesoderm conditioning (Day 11), spin-EBs were collected and analyzed by flow cytometry to assess hematopoietic progenitor cell potential (see flow cytometry methods for antibodies used) (FIG. 4A). Spin-EBs yielding >30% CD34⁺CD45⁺ cells were transferred onto 24-well plates coated with irradiated OP9-DL1 cells (as described in Martin et al. Blood. 2008; 112(7):2730-7; La Motte-Mohs et al. Blood. 2005; 105(4):1431-9) (now defined as Day 11+0). EBs and OP9-DL1 were co-cultured in NK differentiation media (NKDM) supplemented initially with SCF, IL-15, IL-7, Flt3-L (all R&D Systems), and IL-3 (PeproTech) for one week; DMSO, SR-1, or TCDD were also added at Day 11+0. Every week, a one-half media change with NKDM supplemented with SCF, IL-15, IL-7, Flt3-L, and drugs was performed. hESCs were differentiated for four additional weeks (Day 11+28) and non-adherent cells were harvested for analysis.

### Hematopoietic Colony-Forming Unit (CFU) Assay

Day 6+5 spin-EB non-adherent fractions were resuspended in IMDM. 50,000 cells were seeded in 2 mL of H4436 Methocult (StemCell Technologies, Vancouver, CAN) and plated directly in 35 mm culture dishes (Greiner, Monroe, N.C.). Plates were incubated for 14 days and subsequently counted and phenotypically scored using standard criteria (Kaufman et al. Proc. Natl. Acad. Sci. U.S.A. 2001; 98(19):10716-21).

CRISPR-Cas9 Gene Editing and hESC Transfection

gRNA against AHR exon 1 (5′-TCAGATTGTCCCTGGAGGTC-3′ (SEQ ID NO:9) driven by U6 promoter was subcloned into a pCR4-TOPO vector (ThermoFisher Scientific). Single-cell cell adapted H9 RUNX1c-tdTomato reporter cell lines as described in Ferrell et al. Stem Cells. 2015; 33(4):1130-41 were transfected with 1 μg plasmid DNA, 1 μg Cas9 mRNA (TriLink Biotechnologies, San Diego, Calif.), and mCherry fluorescent protein mRNA using the Neon Transfection System (ThermoFisher Scientific) set at 1100V, 20 ms, 1 pulse. Post-transfection, cells were resuspended in MEF conditioned media without antibiotics supplemented with 5 μM Y-27632 and seeded onto Matrigel-coated 6 well plates. 96-hour post-transfection, individual mCherry⁺ colonies were picked onto fresh MEFs for clonal expansion. Genomic DNA was isolated using the DNeasy Blood and Tissue Kit (Qiagen) and AHR PCR products were generated with high fidelity AccuPrime Taq DNA Polymerase (ThermoFisher Scientific). PCR products were purified and subcloned into a pCR-TOPO4 vector for sequencing. Cloned products were transformed into One Shot TOP10 competent cells (ThermoFisher Scientific) and were colony sequenced via rolling circle amplification (Sequetech, Mountain View, Calif.) using M13R primers. On- and off-target effects were assessed using a Surveyor mutation detection kit (IDT Technologies, Coralville, Iowa) and the following AHR-specific primers: F: 5′-AGGCAGCTCACCTGTACT-3′ (SEQ ID NO:10); R: 5′: CATCTCGCCTTACCAAACTCTAC-3′ (SEQ ID NO:11). Only clones that displayed AHR specific cleavage products and had AHR Exon 1 specific deletions as determined by sequencing were chosen for experiments.

### Flow Cytometry

The following additional antibodies were used (all anti-human): LFA-1 (CD11a/CD18)-APC-R700 (BD Biosciences), CD31-APC (eBioscience), CD33-APC (BD Biosciences), CD34-PECy7 (BD Biosciences), CD34-APC (BD Biosciences), CD41a-APC (BD Biosciences), CD43-APC (BD Biosciences), CD45-APC (BD Biosciences), CD56-PECy7 (BD Biosciences), CD56-BV421 (BD Biosciences), CD94-PerCP-Cy5.5 (BD Biosciences), CD117-PECy7 (BD Biosciences), CD117-APC (eBiosciences), CD144-APC (eBioscience). Samples were analyzed on either an LSRFortessa or LSRII flow cytometer (BD Biosciences). Gating was set relative to isotype controls of identical fluorophores. Data from flow cytometry was analyzed using FlowJo software (Treestar, Ashland, Oreg.).

### Cell Cycle Analysis and Proliferation Studies

Spin EBs were differentiated into hemato-endothelial cells with non-adherent cells harvested on Day 6+5. Cells were cultured in a 24-well plate and harvested after 1 week for flow cytometry analysis. Cells were co-stained with anti-human CD34-PECy7 to monitor proliferation of HSPCs. For cell cycle analysis, Day 6+5 non-adherent cells derived from EBs were harvest and monitored for S-phase using the Click-iT EdU Alex Fluor 647 flow cytometry assay kit per manufacturer instructions. Prior to flow cytometry, cells were counterstained with 50 μg/mL propidium iodide supplemented with 100 μg/mL RNAse A (New England BioLabs, Ipswich, Mass.).

RNA Isolation and Quantitative Real-Time PCR (qRT-PCR) Analysis

Non-adherent cell fractions were harvested as previously described in the main text. Total RNA was harvested using the Qiashredder tissue homogenizer kit (Qiagen, Valencia, Calif.) with RNAeasy Mini Kit. RNA concentration and purity was assessed using a NanoDrop 2000 (ThermoFisher Scientific) spectrophotometer and subsequently revere transcribed with a SensiFAST cDNA Synthesis Kit (Bioline, Taunton, Mass.). qPCR was performed using the All-in-One qPCR Mix (GeneCopoeia, Rockville, Md.) and cDNA was used at 50 ng/uL per reaction for qRT-PCR using gene specific human primer pairs (Table 1). qPCR reactions were run on an StepOnePlus Real-Time PCR System thermocycler (ThermoFisher Scientific) in either technical duplicate or triplicate. Cₜ values were normalized to a GAPDH housekeeper for each sample. Data were then normalized relative to controls using the 2̂-(ΔΔCt) method.

TABLE 1
Oligonucleotide primers used for qRT-PCR.

| | | Amplicon | Tm |
|---|---|---|---|
| Gene | Primer Sequence | Size (bp) | (° C.) |
| AHR | F: 5′-CTTAGGCTCAGCGTCAGTTAC-3′ (SEQ ID NO: 12) | 79 | 60 |
| | R: 5′-CGTTTCTTTCAGTAGGGGAGGAT-3′ (SEQ ID NO: 13) | | 61 |
| CYP1A1 | F: 5′-TCGGCCACGGAGTTTCTTC-3′ (SEQ ID NO: 14) | 141 | 62 |
| | R: 5′-GGTCAGCATGTGCCCAATCA-3′ (SEQ ID NO: 15) | | 63 |
| CYP1B1 | F: 5′-AAGTTCTTGAGGCACTGCGAA-3′ (SEQ ID NO: 16) | 142 | 63 |
| | R: 5′-GGCCGGTACGTTCTCCAAAT-3′ (SEQ ID NO: 17) | | 63 |
| OCT4 | F: 5′-GCAGCTCGGAAGGCAGAT-3′ (SEQ ID NO: 18) | 135 | 62 |
| | R: 5′-TGGATTTTAAAAGCGAGAAGACTTG-3′ (SEQ ID NO: 19) | | 62 |
| CMYB | F: 5′-gtcacaaattgactgttacaacaccat-3′ (SEQ ID NO: 20) | 212 | 59 |
| | R: 5′-ttctactagatgagagggtgtctgagg-3′ (SEQ ID NO: 21) | | 60 |
| GATA1 | F: 5′-gggatcacactgagcttgc-3′ (SEQ ID NO: 22) | 176 | 64 |
| | R: 5′-acccctgattctggtgtgg-3′ (SEQ ID NO: 23) | | 65 |
| GATA2 | F: 5′-gcgtctccagcctcatctt-3′ (SEQ ID NO: 24) | 226 | 61 |
| | R: 5′-ggaagagtccgctgctgtag-3′ (SEQ ID NO: 25) | | 60 |
| PU.1 | F: 5′-cacagcgagttcgagagctt-3′(SEQ ID NO: 26) | 194 | 61 |
| | R: 5′-gatgggtactggaggcacat-3′ (SEQ ID NO: 27) | | 61 |
| E4BP4 | F: 5′-GCAGAGCCGATGGAATTAG-3′ (SEQ ID NO: 28) | 222 | 56 |
| | R: 5′-ATCAGTTTCCGACGTTCTCA-3′ (SEQ ID NO: 29) | | 57 |
| EOMES | F: 5′-TCAGATTGTCCCTGGAGGTC-3′ (SEQ ID NO: 30) | 207 | 58 |
| | R: 5′-AGTTTGTTGGTCCCAGGTTG-3′ (SEQ ID NO: 31) | | 58 |
| ID2 | F: 5′-CGGATATCAGCATCCTGTCC-3′ (SEQ ID NO: 32) | 100 | 58 |
| | R: 5′-TCATGAACACCGCTTATTCAG-3′ (SEQ ID NO: 33) | | 56 |
| T-BET | F: 5′-GATGTTTGTGGACGTGGTCTTG-3′ (SEQ ID NO: 34) | 76 | 60 |
| | R: 5′-CTTTCCACACTGCACCCACTT-3′ (SEQ ID NO: 35) | | 61 |
| GATA3 | F: 5′-GCCCCTCATTAAGCCCAAG-3′ (SEQ ID NO: 36) | 80 | 58 |
| | R: 5′-TTGTGGTGGTCTGACAGTTCG-3′ (SEQ ID NO: 37) | | 60 |
| RORγt | F: 5′-GCCAAGGCTCAGTCATGAGAA-3′ (SEQ ID NO: 38) | 61 | 60 |
| | R: 5′-TTGTCCCCACAGATTTTGCA-3 ′ (SEQ ID NO: 39) | | 58 |
| IL1R1 | F: 5′:cctgctatgattttctcccaataaa-3′ (SEQ ID NO: 40) | 115 | 57 |
| | R: 5′-aacacaaaaatatcacagtcagaggtagac-3′ (SEQ ID NO: 41) | | 62 |
| IL22 | F: 5′-GCTTGACAAGTCCAACTTCCA-3′ (SEQ ID NO: 42) | 140 | 59 |
| | R: 5′-GCTCACTCATACTGACTCCGTG-3′ (SEQ ID NO: 43) | | 60 |
| GAPDH | F: 5′-ccactcctccacctttgac-3′ (SEQ ID NO: 44) | 102 | 58 |
| | R: 5′-accctgttgctgtagcca-3 (SEQ ID NO: 45) | | 58 |

### Immunoblotting

Undifferentiated hESCs were harvested and lysed in lysis buffer (10 mM HEPES pH 7.4, 150 mM KCl, 0.1% NP40, 5 mM MgCl₂ supplemented with EDTA-free protease inhibitor tablet (Roche, Branchburg, N.J.)) overnight at 4° C. Protein concentration was determined by bicinchoninic acid (BCA) assay, per manufacturer protocol (ThermoFisher Scientific). 50 μg of protein lysate was added to 6×SDS loading buffer (250 mM Tris, 2% SDS, 20% glycerol, 0.05% bromphenol blue), loaded onto a 4-20% Mini-PROTEAN TGX Gel (BioRad, Hercules, Calif.), and electrophoresed at 120V for 1 hour. Protein was then transferred to a nitrocellulose membrane in transfer buffer (25× Tris-Glycine transfer buffer with 5×100% methanol) overnight at 30V. Membranes were blocked with 5% milk for 1 hour at room temperature and subsequently incubated with the following primary antibodies overnight at 4° C. in blocking buffer (all human): mouse monoclonal anti-AHR (ab2769; Abcam, Cambridge, Mass.), rabbit polyclonal anti-AHRR (ab108518; Abcam), rabbit polyclonal anti-CYP1B1 (ab137562; Abcam), or rabbit monoclonal anti-actin (Sigma-Aldrich, St. Louis, Mo.). Membranes were washed three times with PBST and incubated with either anti-rabbit or anti-mouse IgG HRP-link secondary antibody (Cell Signaling Technologies, Danvers, Mass.). Bands were visualized on film using SuperSignal West Dura Extended Duration Substrate (ThermoFisher Scientific) and standard film processor at various exposure times.

### CD107a Degranulation Assay

To examine NK cell activity, as previously described (Ng et al. Nat. Protoc. 2008; 3(5):768-76; Tian et al. Stem Cells. 2009; 27(11):2675-85; Bai et al. J. Cell. Biochem. 2010; 109(2):363-374; Bai et al. J. Cell. Physiol. 2016; 231(5):1065-1076), hESC-derived NK cells were harvested, washed, and plated at 100,000 cells per well of a V-bottomed 96-well plate co-cultured with K562 at a 2:1 effector-to-target ratio. NK cell only and NK+K562 wells were incubated with anti-CD107a-AF700 (BD Biosciences, San Jose, Calif.) for 1 hour. Following incubation, 1:1500 Golgi Stop and 1:1000 Golgi Plug mix (BD Biosciences) was added and cells were incubated for 4 hours. Cells were washed with PBS and counterstained with Near-IR Live/Dead Fixable Dye (ThermoFisher Scientific). Cells were washed again and co-stained with the following antibodies (all anti-human): CD3-PE-Texas Red (eBioscience, San Diego, Calif.), CD14 PE-Texas Red (eBioscience), CD19-PE-Texas Red (eBioscience), and CD56-PE (Beckman Coulter, Indianapolis, Ind.) for 30 minutes at 4° C. Cells were then transferred to FACS tubes and analyzed.

Fluorescent Activated Cell Sorting (FACS) of NKP, cNK, and ILC3 Derived from hESCs

To assess the effects of AHR modification on NK cell and ILC specification, non-adherent hematopoietic cells derived from hESCs on Day 11+28 cultured in NKDM supplemented with DMSO, SR-1, or TCDD were harvested as single-cell suspensions. Cells were washed with DPBS, filtered, and stained with the following antibodies (all anti-human): CD56-BV421, CD94-PerCP-Cy5.5, CD117-PECy7, and LFA-1 (CD11a/CD18)-APC-R700 in sterile FACS Buffer (DPBS+2% FBS and 0.1% sodium azide) for 30 minutes at 4° C. Cells were washed with FACS buffer and cells were counterstained with Sytox Blue (ThermoFisher Scientific) immediately prior to sorting. Live NKP, cNK, and ILC3 populations were sorted directly into FACS tubes containing NKDM basal media using a FACSAria II (BD Biosciences) cell sorter. Cells were immediately centrifuged, washed once with PBS, and processed from total RNA isolation as described above.

### Statistical Analyses

Differences between groups were compared either with student's t-test or one-way/two-way ANOVA using Prism 6 (GraphPad Software, San Diego, Calif.). Results were considered statistically significant at p-values <0.05.

### Results

Small Molecule Antagonism of AHR Enhances Early Hemato-Endothelial Differentiation from hESCs

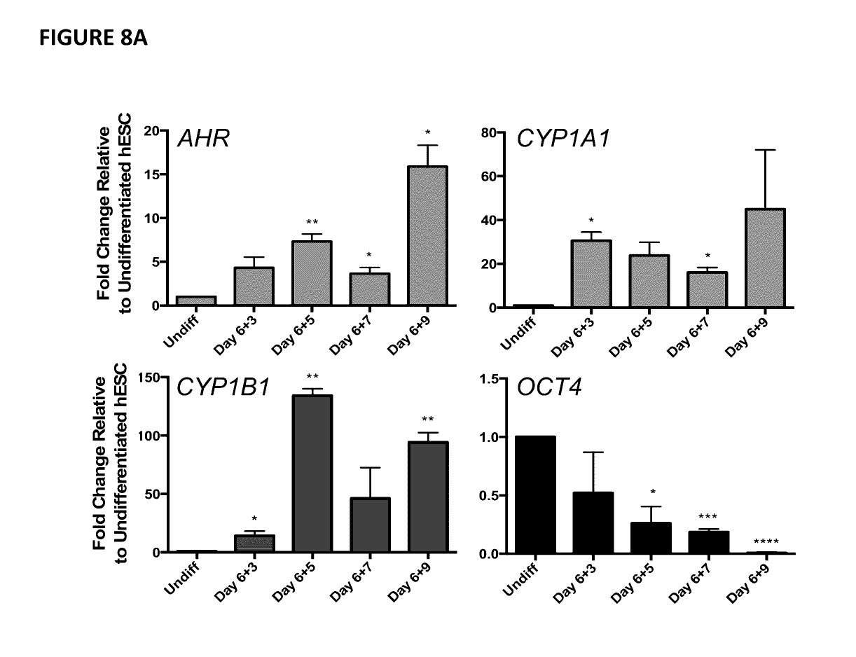

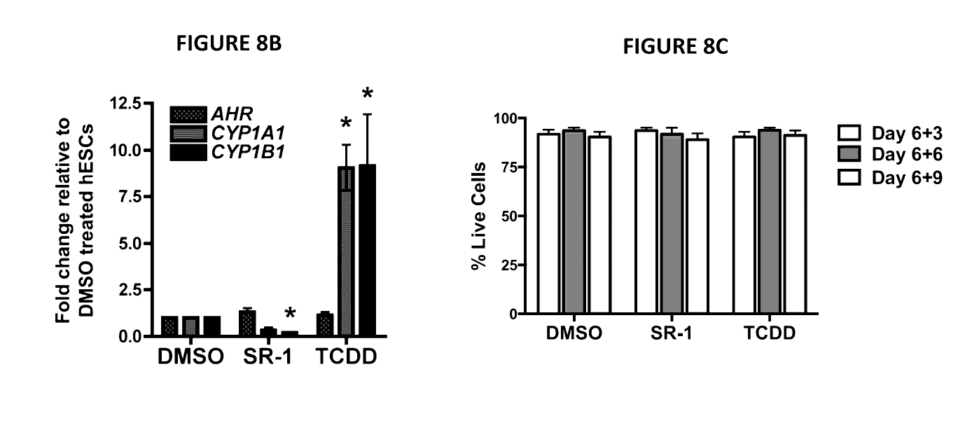

To establish the role of AHR in the development of the earliest human hematopoietic cells, human embryonic stem cells (hESCs) were differentiated using a two-stage defined culture system, as previously described (Ferrell et al. Stem Cells. 2015; 33(4):1130-41; Ng et al. Nat. Protoc. 2008; 3(5):768-76) (FIG. 7A). AHR expression in undifferentiated hESCs and hESCs differentiating into hemato-endothelial cells was first determined under these defined conditions. AHR expression was increased 4.30±1.24 fold in the differentiated cell population at Day 6+3 relative to undifferentiated hESCs and became significantly increased at Day 6+5 (7.33±1.24 fold, p<0.01) (FIGS. 7A, 8A). A corresponding increase in the expression of two downstream effector targets of AHR signaling (CYP1A1 and CYP1B1) was also observed. These data indicate that endogenous AHR activity is upregulated at the onset of hemato-endothelial differentiation from hESCs, suggesting that AHR is implicated in early hematopoiesis. hESCs were next treated with a DMSO vehicle control or with SR-1 or TCDD to modulate AHR signaling. Following 4 days of culture, hESCs treated with 1 μM SR-1 had reduced expression of CYP1A1 and CYP1B1, whereas hESCs treated with TCDD yielded a significantly increased expression of CYP1A1 and CYP1B1 as compared to DMSO normalized controls. There were no significant cytotoxic effects on hESCs due to the presence of SR-1 or TCDD (FIGS. 7C, 8C). Together, SR-1 and TCDD can effectively be used as agents to selectively regulate AHR-mediated activity in hESCs.

Differentiating hemato-endothelial cells when exposed to SR-1 or TCDD were also investigated. As early as Day 6+3, there was a marked increase in the total percentage of CD34⁺CD144⁺ (1.71±0.22 fold, p<0.05) and CD34⁺CD31⁺ (1.58±0.28 fold) cells that have dual hemato-endothelial cell developmental potential in SR-1 treated hESCs as compared to DMSO controls (FIGS. 7B & 7C). As differentiation continued to Day 6+6, there were also notable increases in the total percentage of both CD34⁺CD31⁺ (1.62±0.12 fold, p<0.05) hemato-endothelial cells and budding CD34⁺CD43⁺ (1.36±0.12 fold) hematopoietic progenitor cells when SR-1 treatment was applied. At Day 6+9, there was a significant increase in development of CD34⁺CD45⁺ hematopoietic progenitor cells (1.40±0.11, p<0.05) in the SR-1 treated cells. Development of more terminally differentiated hematopoietic phenotypes (CD34⁻CD43⁺ and CD34⁻CD45⁺) was also observed when hESCs were treated with TCDD. This effect was most pronounced at the Day 6+9 time point, where there was a reduction in the total percent of CD34⁺CD144⁺ (0.33±0.13), CD34⁺CD31⁺ (0.49±0.06, p<0.05), CD34⁺CD43⁺ (0.67±0.09, p<0.05), and CD34⁺CD45⁺ (0.44±0.06, p<0.05) progenitor cells with an increase in the total percent of CD34⁻ hematopoietic cells. Taken together, these data demonstrate that AHR inhibition with SR-1 promotes early hemato-endothelial cell development, whereas AHR hyperactivation with TCDD accelerates differentiation towards more terminally differentiated hematopoietic lineages.

AHR-Modulation in hESC-Derived Hemato-Endothelial Cells Alters Cell Cycle Progression

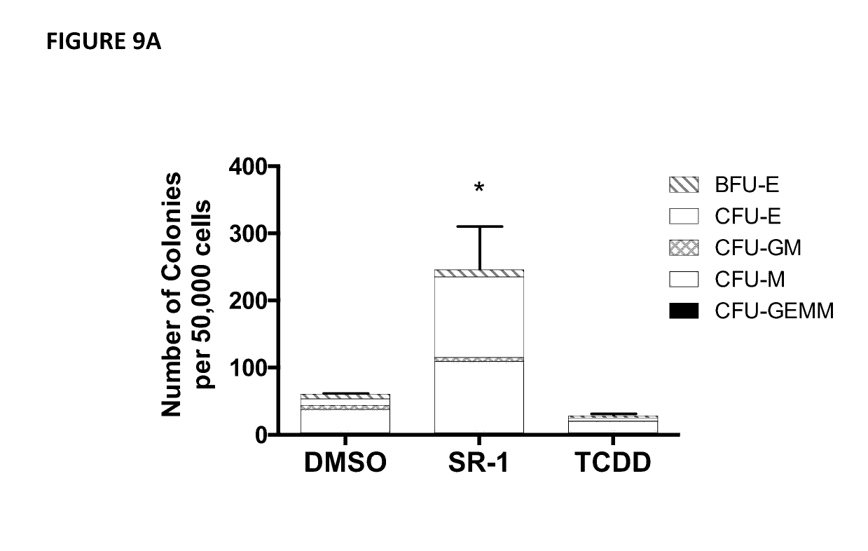

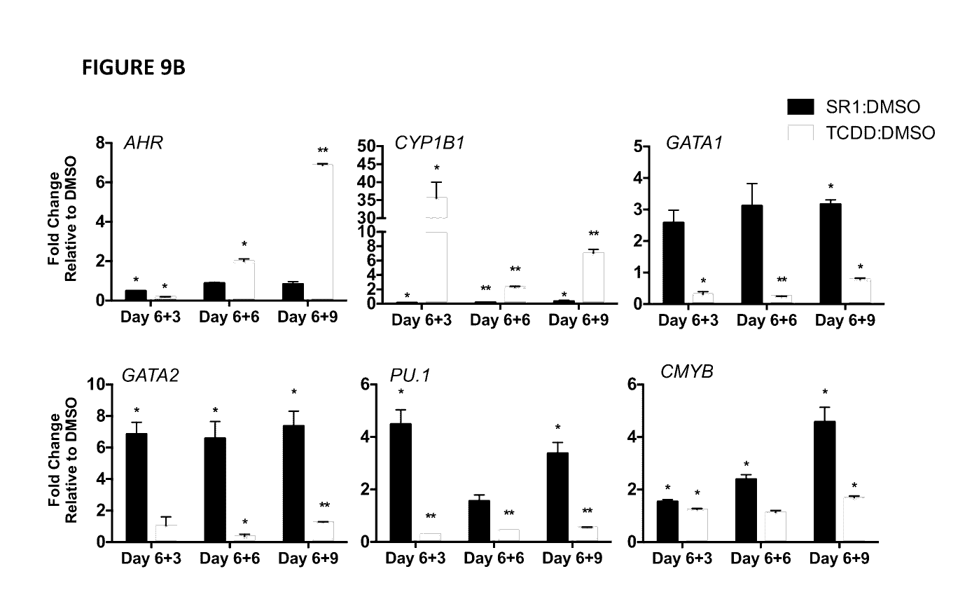

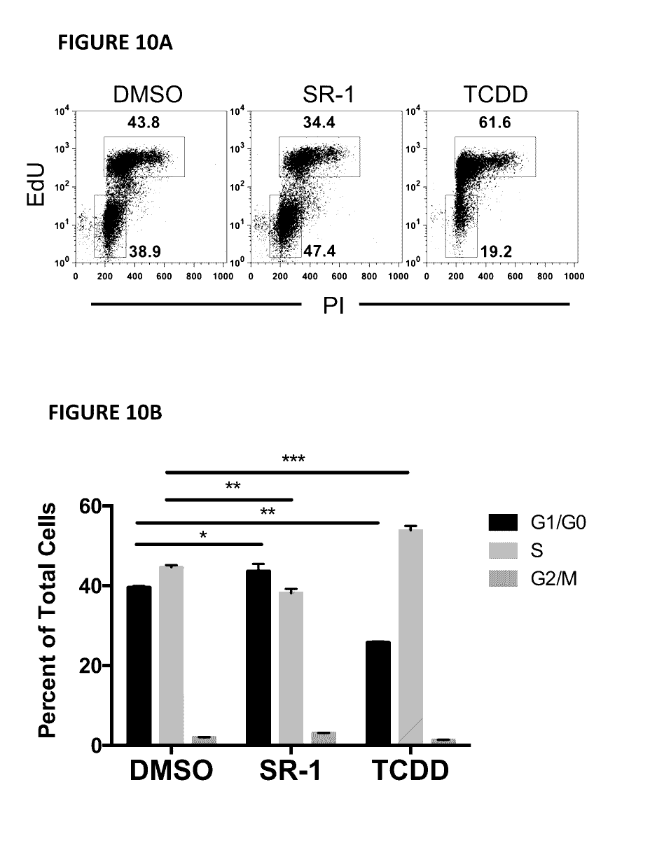

Next, whether the increased hemato-endothelial phenotype seen with SR-1 mediated inhibition of AHR was due to alterations in cell cycle progression was investigated. hESC-derived, non-adherent hematopoietic progenitor cells treated with either DMSO, SR-1, or TCDD at Day 6+5 were analyzed to assess for the percentage of cells in G₀/G₁ (EdU⁻PI^{low}), S-phase (EdU⁺PI⁺), and G₂/M (EdU⁻PI^{hi}) (FIGS. 9A, 10A). SR-1 treated hematopoietic progenitor cells had a significant increase for G₀/G₁ phase (43.7%±1.82, p<0.05) rather than S-phase (38.2%±1.00, p<0.01) as compared to DMSO treated controls (39.6%±0.35 and 44.6%±0.48, respectively) (FIG. 9B, 10B). Conversely, TCDD treated hematopoietic progenitor cells were significantly enriched for S-phase (38.2%±1.03, p<0.001) rather than G₀/G₁ phase (25.8±0.34, p<0.01) as compared to the same controls. Collectively, these data demonstrate AHR regulates hemato-endothelial and hematopoietic progenitor cell phenotype via cell cycle modification.

### AHR Inhibition Leads to Functional Hematopoietic Progenitor Cells and Increased Expression of Key Hematopoietic Genes

Whether SR-1 supported the production of functional hematopoietic progenitor cells was assessed by standard methylcellulose-based colony forming unit (CFU) assays. SR-1 conditioning led to a marked increase in hematopoietic progenitor cell development compared to DMSO-treated controls (245.67±64.4 colonies vs. 60.3±1.20 colonies, respectively, p<0.05), while the TCDD-treated cells were significantly decreased (28.3±2.67 colonies, p<0.05) (FIG. 9A).

Key transcriptional regulators of human hematopoiesis that may be modulated by AHR expression within developing hemato-endothelial cells were also assessed. The non-adherent hematopoietic fractions of differentiating hESC-derived cells treated with DMSO, SR-1, and TCDD were analyzed and qRT-PCR probing for AHR-related genes (AHR, CYP1B1), megakaryotic-erythropoietic genes (GATA1 and GATA2), a myelopoiesis regulator (PU.1), and a definitive hematopoiesis specific gene (CMYB) was performed. SR-1 treatment increased the expression of GATA1 (2.58±0.40 fold) and GATA2 (6.85±0.74 fold, p<0.05) as early as Day 6+3 (FIG. 9B). The mean GATA2:GATA1 at Day 6+3 was 2.67, and this positive ratio is in accord with the elevated GATA2 endogenous gene progression relative to GATA1 throughout early erythropoiesis. TCDD treatment decreased the expression of GATA1 at Day 6+3 (0.31±0.08 fold, p<0.05) as compared to DMSO controls and induced a reduction in GATA2 later at Day 6+6 (0.37±0.13 fold, p<0.05). There was a similar induction of PU.1 with SR-1 treatment and reciprocal expression in TCDD treated hematopoietic cells at each time point. The increased fold-change of GATA1/GATA2 and PU.1 expression supports the enhanced production of CFU-E and CFU-M in SR-1 treated hematopoietic progenitor cells (FIG. 9A). Moreover, SR-1 treatment also mediated a significant increase of CMYB at all time points. Collectively, these results further demonstrate AHR inhibition leads to enhanced activation of a functional and multilineage hematopoietic transcriptional program from hESCs.

AHR Knockout in hESCs Promotes Early Hematopoietic Differentiation

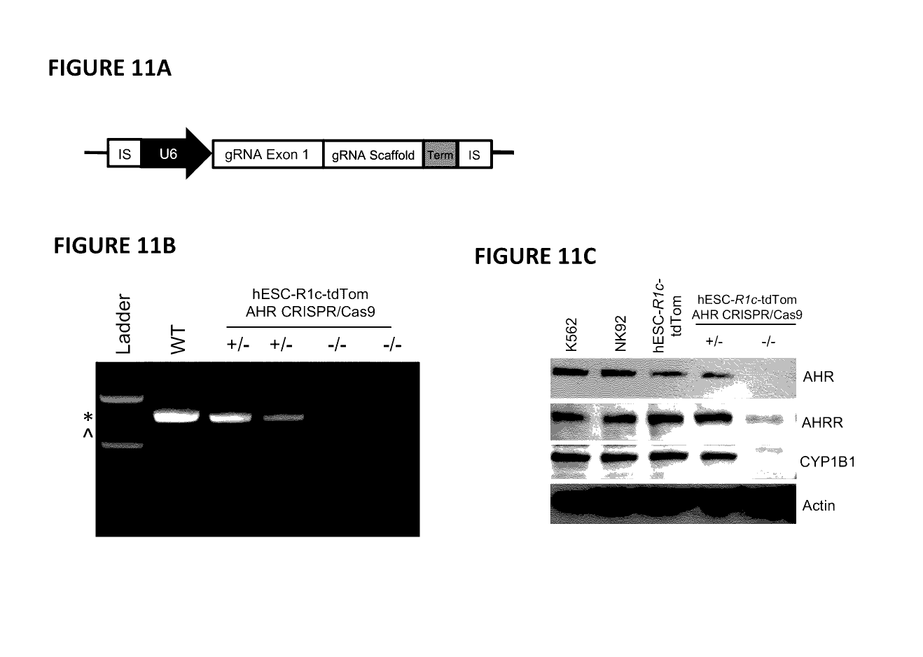

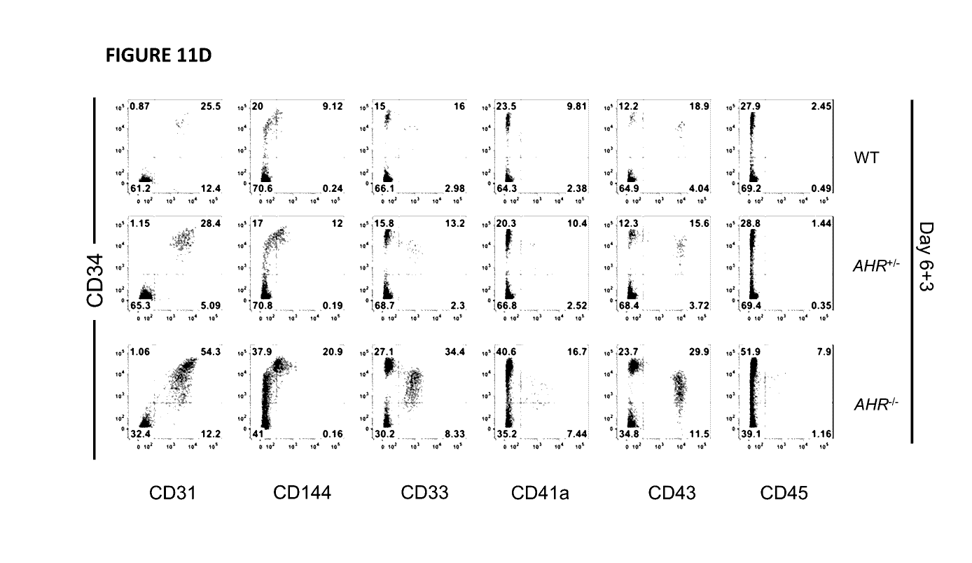

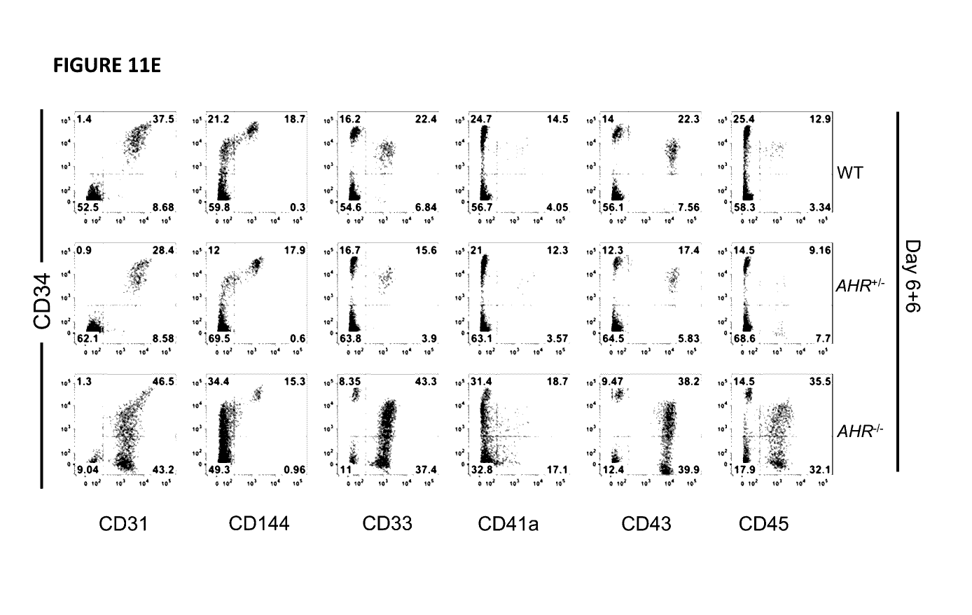

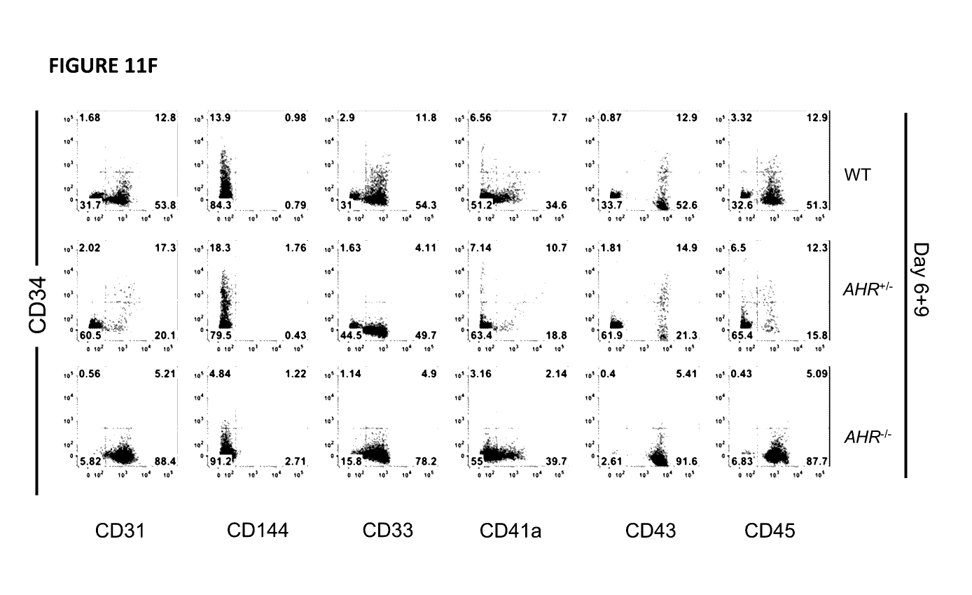

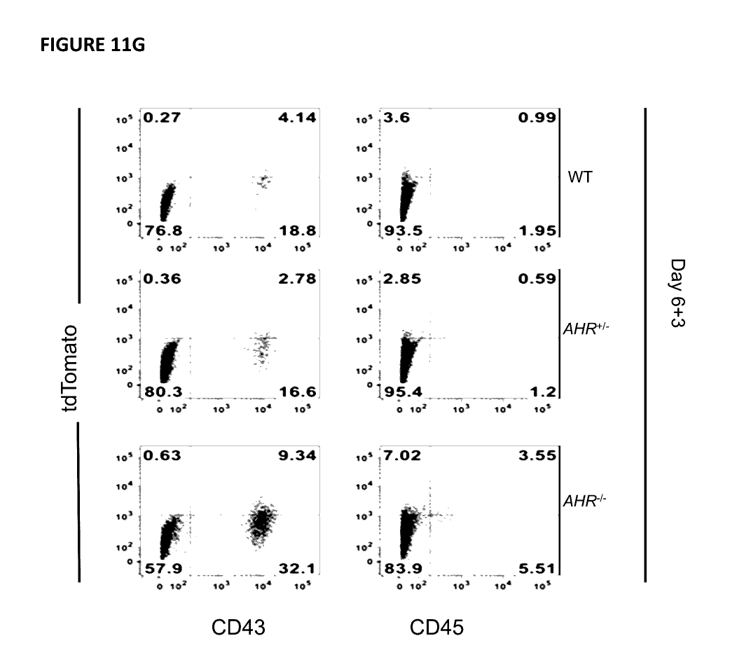

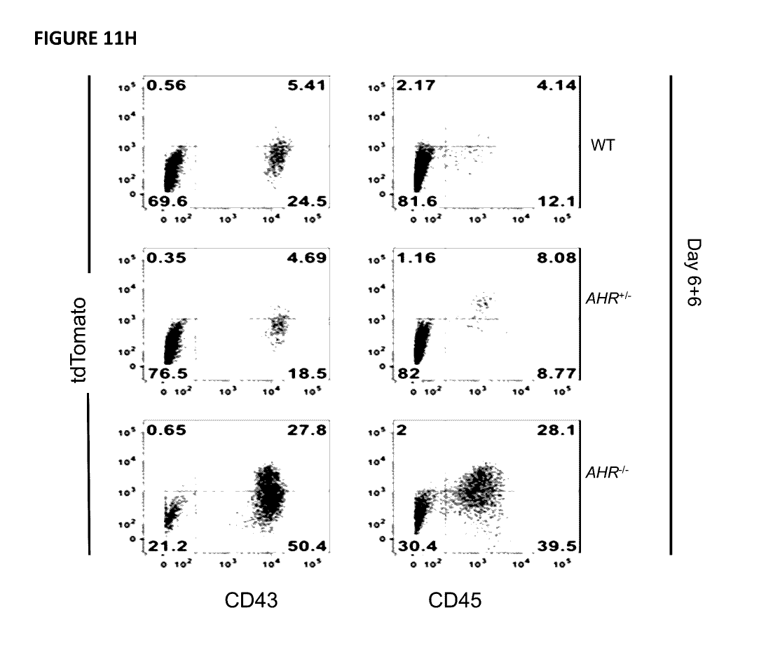

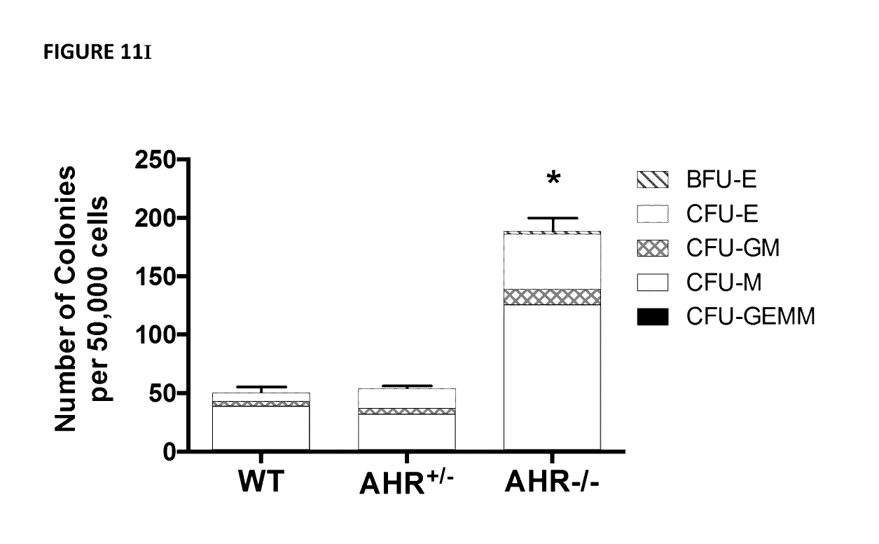

CRIPSR/Cas9-mediated gene deletion provides a more targeted approach to define the role of AHR during early human hemato-endothelial and hematopoietic progenitor cell production. CRIPSR/Cas9 was used to develop stable and clonally-derived hESCs cell lines with a targeted AHR deletion. Specifically, hESCs previously modified with a RUNX1c-tdTomato reporting cassette that demonstrates faithful measurement of early hemato-endothelial cells (Ferrell et al. Stem Cells. 2015; 33(4):1130-41) were used. These cells allowed the observation of EHT and the isolation of early human hematopoietic cells as they emerge from adherent endothelial cells. These cells allow the dual evaluation of the effect of AHR gene modification on the induction of EHT. Clonally expanded hESCs were transfected with a gRNA target complementary to AHR exon 1 and probed for modification using primers flanking the exon 1 sequence (FIG. 11A). Clones that yielded a 718 base pair (bp) amplicon (wild-type, WT), a 718 bp amplicon with an additional 571 bp amplicon indicative of partial exon 1 deletion (AHR^{+/-}), and only the 571 bp amplicon (AHR^{-/-}) were identified (FIG. 11B). Functional loss of AHR protein with significant attenuation of the AHR-downstream genes aryl hydrocarbon receptor repressor (AHRR) and CYP1B1 in AHR^{-/-}-hESC-RUNX1c-tdTomato cells, as compared to K562 and NK92 positive controls, and wild-type hESC-RUNX1c-tdTomato cells was confirmed (FIG. 11C). The on-target specificity of the gRNA was validated by probing the AHR amplicons generated from the genomic DNA of each clone with Surveyor endonuclease as well as with direct sequencing. These data confirmed successful generation of heterozygous and homozygous deletions of AHR within hESCs.

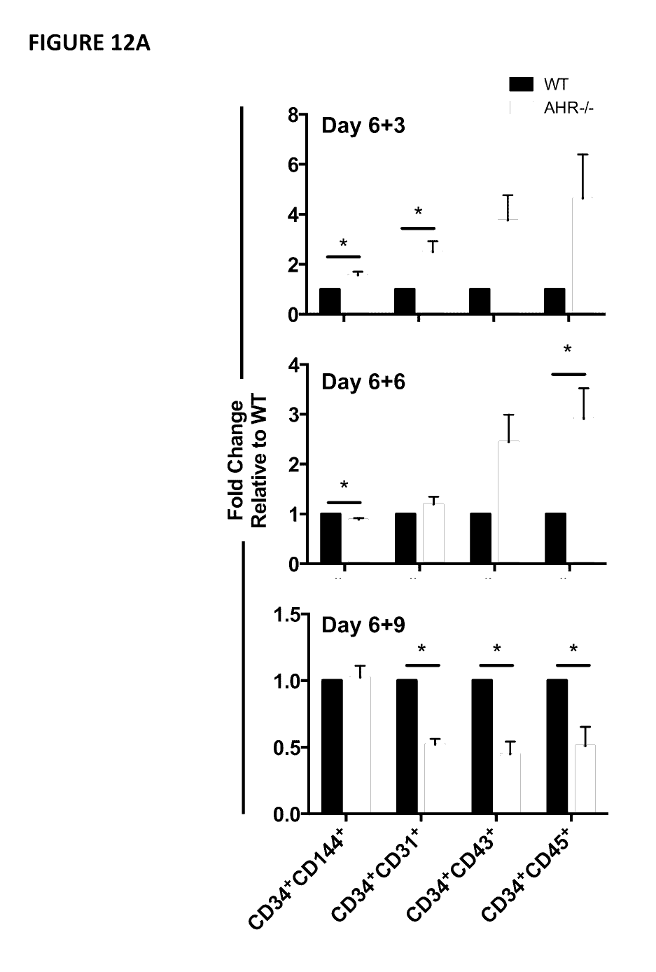

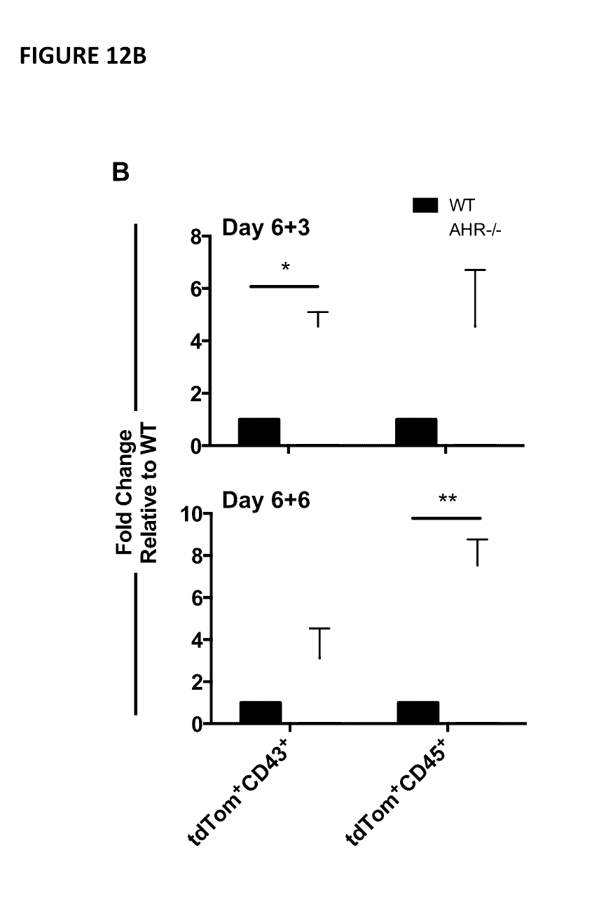

WT-, AHR^{+/-}-, and AHR^{-/-}-RUNX1c-tdTomato hESCs were differentiated as in previous studies. At Day 6+3, there was approximately a 2-fold increase in development of hemato-endothelial cells (CD34⁺CD31⁺ and CD34⁺CD144⁺) as compared to WT- and AHR^{+/-}-hESCs at Day 6+3 (FIG. 11D, quantified in FIGS. 11A, 12A). AHR^{-/-}-RUNX1c-tdTomato hESCs produced more than a 2-fold increase in CD34⁺CD43⁺ and CD34⁺CD45⁺ hematopoietic progenitor cells at both Day 6+3 and Day 6+6 time points. The total percentage of CD34⁺was not compromised as hematopoietic progenitor cells further differentiated into mature hematopoietic cells (CD34⁻CD33⁺, CD34⁻CD41a⁺, CD34⁻CD43⁺, CD34⁻CD45⁺). By Day 6+9, a majority of the AHR^{-/-}-hESC-derived cells continued to differentiate into matured hematopoietic lineages at a greater rate than WT- and AHR^{+/-}-hESCs, as indicated by an increased total percentage of CD34⁻CD45⁺ cells.

Using the RUNX1c-tdTomato reporter, an increased commitment towards RUNX1c⁺ cell development in AHR^{-/-}-RUNX1c-tdTomato hESCs as compared to WT- and AHR^{+/-}-RUNX1c-tdTomato hESCs was demonstrated. Specifically, there was a 5-fold expansion in the total percentage of tdTomato⁺ hematopoietic progenitor cells at both Day 6+3 and Day 6+6 in AHR^{-/-}-RUNX1c-tdTomato hESCs compared to WT- and AHR^{+/-}-RUNX1c-tdTomato hESCs (FIG. 11E, quantified in FIGS. 11B, 12B). Increased development of functional hematopoietic progenitor cells derived from AHR^{-/-}-RUNX1c-tdTomato hESCs compared to the controls using hematopoietic colony-forming unit assays was confirmed. There was a significant increase in the total number of colonies formed in the AHR^{-/-}-hESCs (188.67±11.29 colonies, p<0.05) as compared to AHR^{+/-}-hESCs (54.0±2.08 colonies) and WT-hESCs (50.33±4.91 colonies) (FIG. 11F). Collectively, these data demonstrate that genetic deletion of AHR in hESCs mediates a significant increase in functional hemato-endothelial differentiation.

AHR Inhibition Enhances cNK Cell Differentiation from hESCs while AHR Hyperactivation Supports ILC3 Cell Phenotype

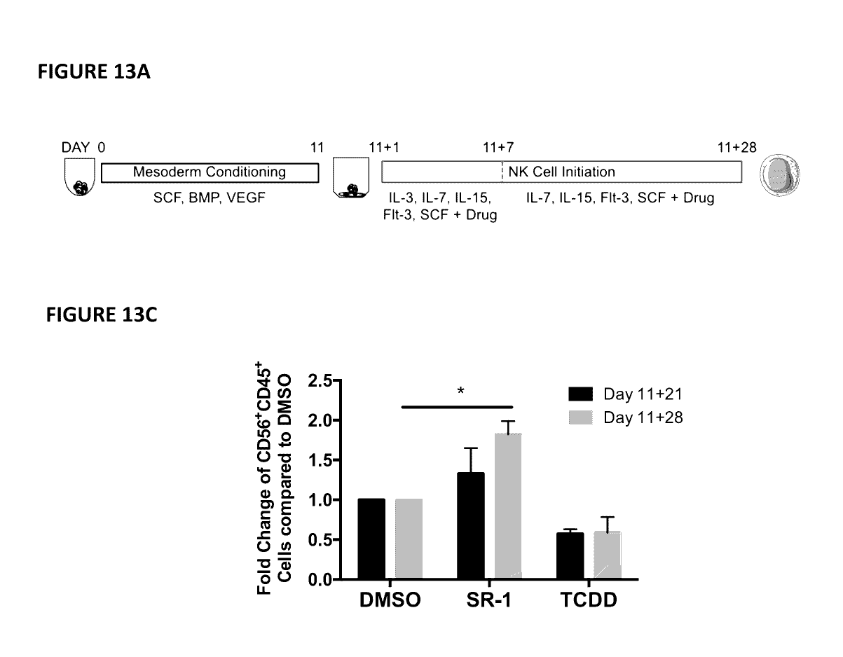

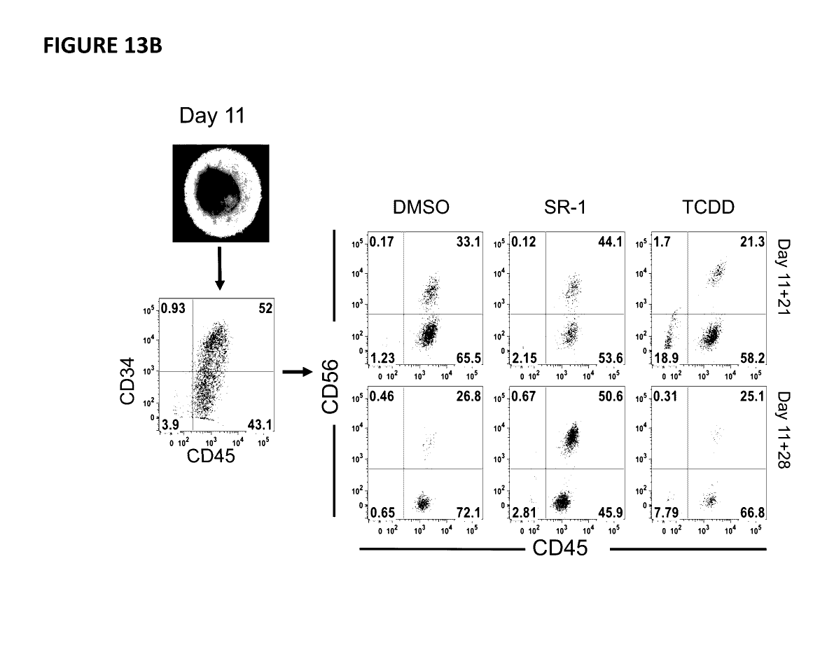

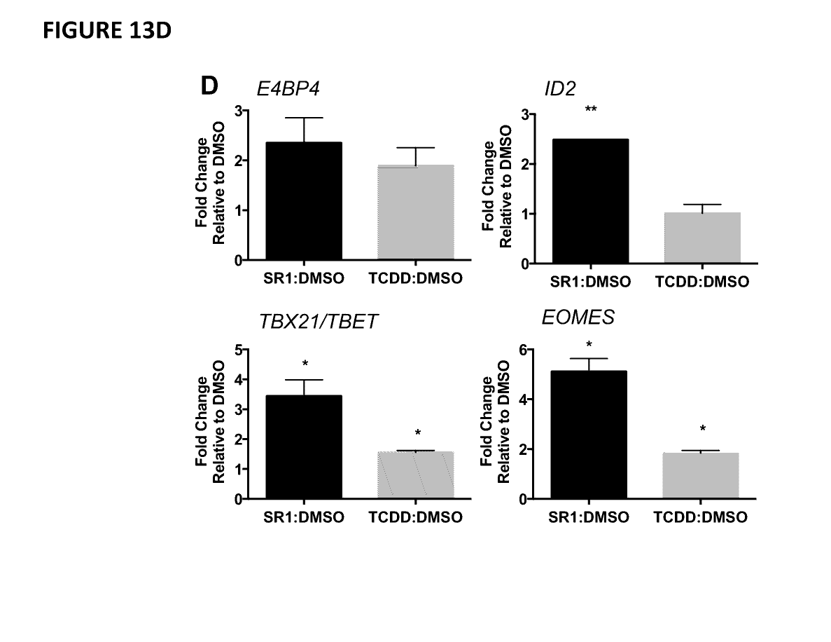

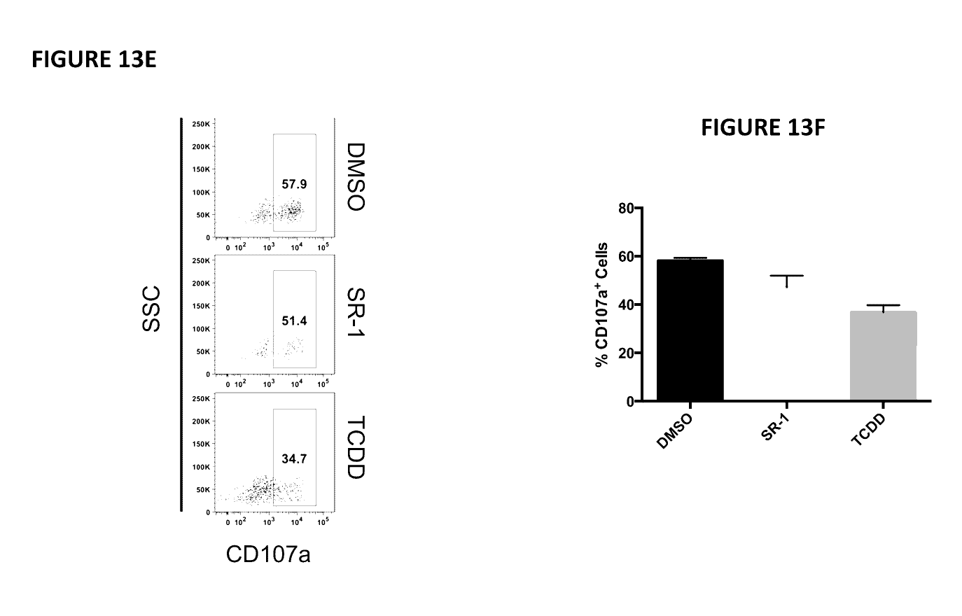

AHR plays a role in mediating development and function of both innate and adaptive immune cells. Since AHR attenuation supports the differentiation of CD34⁺CD45⁺ hematopoietic progenitor cells (FIGS. 7B, 11D), whether NK cell differentiation could also be enhanced from hESCs using defined conditions and a small molecule approach was assessed. Here, a system for NK cell development from hESCs as a model for lymphopoiesis was used (Knorr et al. Stem Cells Dev. 2013; 22(13):1861-9; Ni et al. Embryonic Stem Cell Immunobiol. Methods Protoc. Methods Mol. Biol. 2013; 1029:33-41; Hermanson et al. Stem Cells. 2016; 34(1):93-101) (FIG. 13A). By Day 11, spin-EBs produced a high percentage of CD34⁺CD45⁺ hematopoietic progenitor cells (range: 38.5%-65.0% for n=3 separate studies) (FIG. 13B). At Days 11+21 (11 days in hematopoietic differentiation conditions, then 21 days in NK cell differentiation conditions) and Day 11+28, SR-1 treated hESC-derived hematopoietic cells demonstrated increased development of NK cells compared to DMSO treated controls, while TCDD treated hESC-derived hematopoietic cells had fewer phenotypic NK cells (FIGS. 13B, 13C). In addition to surface antigen acquisition, lymphoid-specific gene expression in the hematopoietic cells produced in each treatment group was assessed. As compared to the DMSO-treated control group, SR-1-treated hESC-derived hematopoietic cells expressed a significantly higher amount of ID2 (2.49±0.003 fold, p<0.01), TBX21/TBET (3.44±0.55 fold, p<0.05), and EOMES (5.12±0.52 fold, p<0.05), transcriptional factors that mediate increased NK cell lineage commitment (FIG. 13D). While a significant increase in TBX21/TBET (1.56±0.07 fold, p<0.05) and EOMES (1.84±0.11 fold, p<0.05) in the TCDD treated hESC-derived hematopoietic cells was observed, the fold-induction was significantly lower than those of the SR-1 treated group. The functionality of differentiated NK cells was assessed via CD107a degranulation when stimulated with K562 target cells. SR-1 treated hESC-derived hematopoietic cells were comparable to DMSO treated controls in CD107a expression (58.1±0.67% vs. 47.2±2.76%), while TCDD treated hESC-derived hematopoietic cells expressed less CD107a (36.8±2.1%) (FIGS. 4E & 4F). Collectively, these data support SR-1 treatment of differentiating hESCs enhances the production of functional NK cells.

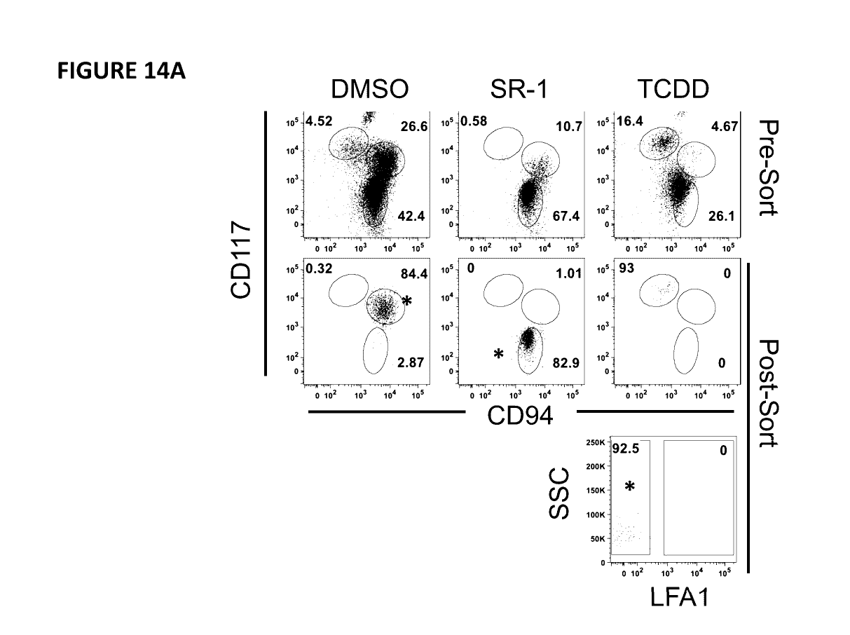

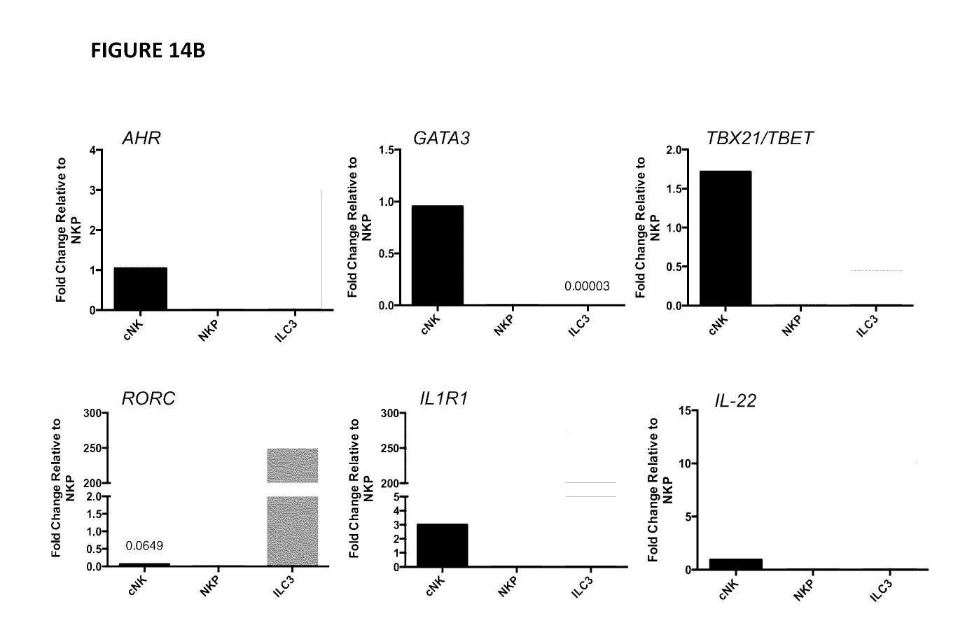

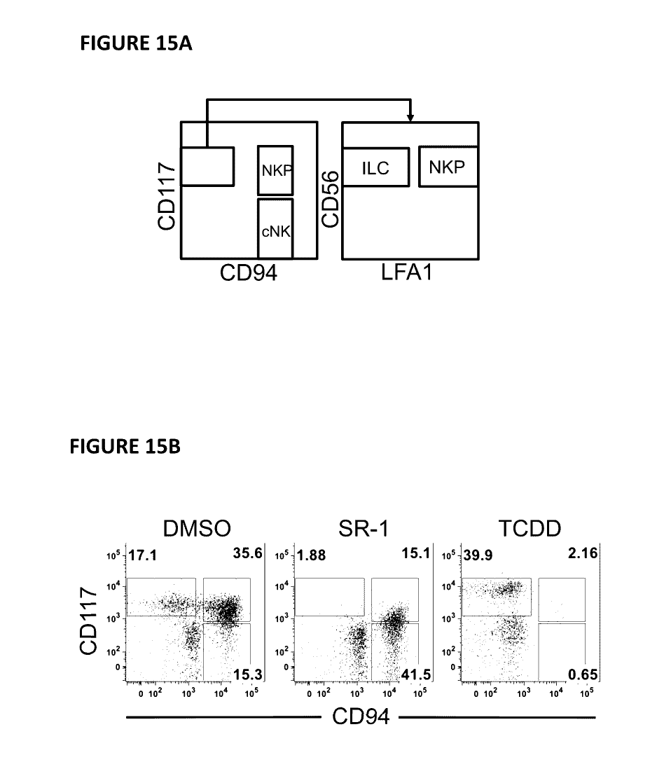

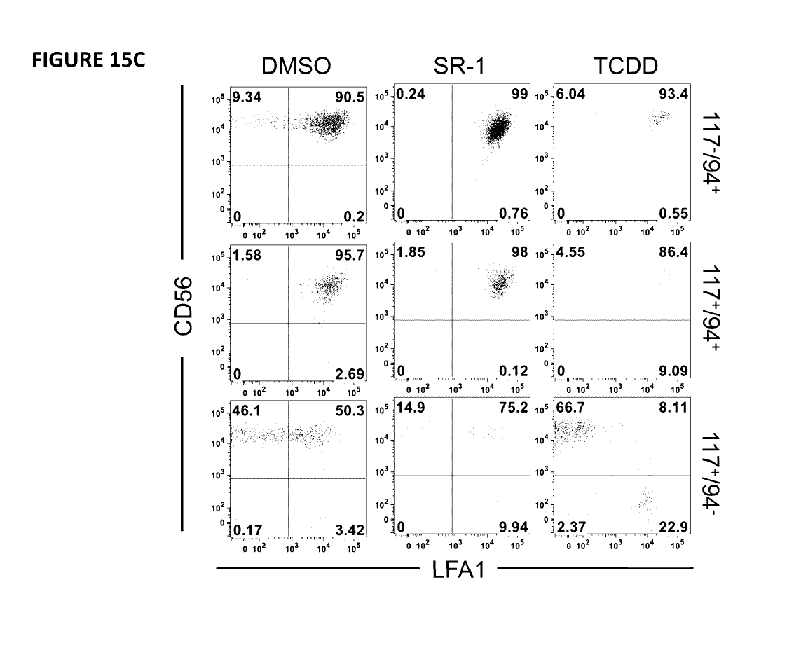

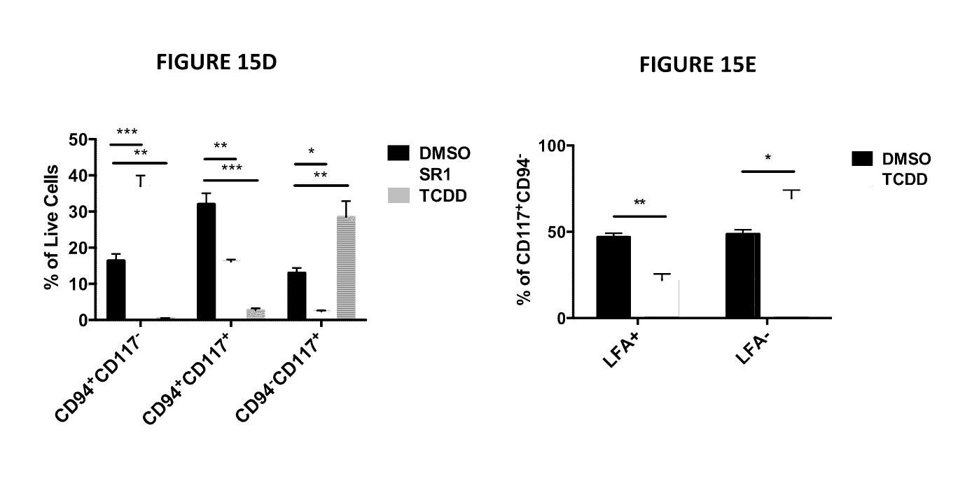

The identity of developing lymphoid phenotypes regulated by AHR activity was further defined by evaluating natural killer progenitor cells (NKP), conventional NK cells (cNK), and developmentally related innate lymphoid group 3 cells (ILC3). At Day 11+28, hESC-derived hematopoietic cells treated with DMSO control produced cNK (CD94⁺CD117⁻CD56⁺LFA1⁺) and ILC3 (CD94⁻CD117⁺CD56⁺LFA1⁻) cells, but with a majority of the differentiated cells restricted to the NKP (CD94⁺CD117⁺) gate (FIGS. 15A, 15B). Treatment with SR-1 significantly shifted hESC-derived hematopoietic cells away from NKPs (16.13±0.58% vs. 32.0±2.98%, p<0.01) and toward cNK cells (37.0±2.92% vs. 16.5±1,77%, p<0.001) compared to DMSO, with a significant reduction in the CD94⁻CD117⁺population (FIG. 15D). Treatment with TCDD also significantly shifted hESC-derived hematopoietic cells away from NKPs and led to reciprocal increase in CD94⁻CD117⁺ cells (28.5±4.42% vs. 13.1±1.34%, p<0.01). When CD94⁻CD117⁺ cells were gated to distinguish the presence of ILC3s, a significantly larger percentage TCDD treated hESC-derived hematopoietic cells were absent for LFA1, as compared to DMSO treated controls (69.4±4.57% vs. 48.8±4.81%, p<0.05) (FIG. 15E). LFA1 expression is a unique and distinguishing marker between ILC3 (LFA1⁻) and cNK (LFA1⁺). Populations of cNK, NKP, and ILC3 were sorted and qRT-PCR was performed for both NK and ILC3 specific gene expression (FIGS. 13A, 14A). As expected, hESC-derived phenotypic ILC3 cells had a classical ILC3 gene signature, specifically RORc, IL1-R1, and IL-22. Additionally, ILC3 sorted populations were virtually deficient for GATA3, a transcriptional regulator of Group 2 ILCs, and displayed decreased TBX21/TBET expression (FIGS. 13B, 14B). These data support the hypothesis that AHR inhibition promotes the differentiation of NK progenitor cells into mature cNK cells. Furthermore, for the first time, AHR hyperactivation was demonstrated to promote development of an ILC3 phenotype from hESCs.

### DISCUSSION

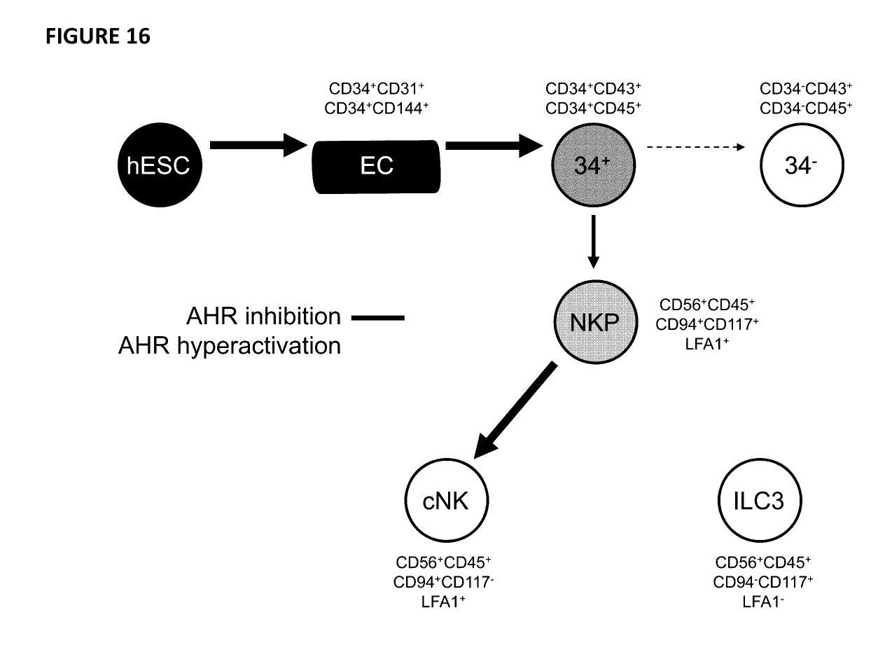

Human pluripotent stem cells provide a starting point to better define key molecular and genetic drivers of human hemato-endothelial development. As described herein, AHR antagonism using the chemical inhibitor SR-1, as well as AHR gene deletion using the CRIPSR/Cas9 system, can enhance human EHT and hematopoietic progenitor cell development. In corresponding fashion, AHR hyperactivation using TCDD can suppress development of hematopoietic progenitor cells with multilineage potential and accelerate their differentiation into more matured hematopoietic lineages (FIG. 16).

Interestingly, AHR gene deletion enhances development of early hematopoietic progenitor cells that are differentiating into definitive hematopoietic lineages. The RUNX1c isoform is correlated with emerging definitive HSPCs from aorta-gonad-mesonephros region endothelial cells. Using a RUNX1c-tdTomato reporter system to model EHT, hESCs harboring AHR gene deletion were found to enhance the differentiation of RUNX1c⁺ hematopoietic cells. An induction of a multilineage transcriptional program, including typical genes expressed during definitive hematopoiesis, was also observed. Without wishing to be bound by theory, this effect may be the result of AHR functioning as a modulator of β-catenin/Wnt signaling.

In addition to increased development of hemato-endothelial cells, differentiation of lymphoid cells (NK cells) was increased by treatment of hESCs with SR-1. The hESC-derived NK cells in the presence of SR-1 were functional, in that they comparably degranulated (CD107a) relative to controls when stimulated with K562 targets. The role of AHR in hemato-lymphoid development was further confirmed by demonstrating AHR antagonism enhances differentiation of NK progenitor cells into cNK phenotypes. This study illustrates that SR-1 can be added into currently defined differentiation protocols to enhance the efficiency and homogeneity of hESC-derived NK cells suitable to human clinical trials.

Finally, this disclosure describes that ILC3s can be derived from human pluripotent stem cells. hESC-derived ILC3, like ILC3 located in secondary lymphoid tissue and peripheral blood, require AHR to drive their development. hESCs, particularly in conjunction with a CRISPR/Cas9 gene editing system, can be used as a powerful platform to better understand the development of a range of human an innate lymphoid cells, as well as to better analyze their effector phenotypes and therapeutic potential.

The complete disclosure of all patents, patent applications, and publications, and electronically available material cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

What is claimed is:

1. A method comprising
inducing hematopoietic differentiation of a human stem cell; and
disrupting aryl hydrocarbon receptor (AHR) activity in the cell,
wherein differentiation of the cell to at least one of a hemato-endothelial cell, a hematopoietic progenitor cell, and a hemato-lymphoid cell is enhanced compared to differentiation without disruption of AHR activity.

2. The method of claim 1, wherein the hemato-endothelial cell comprises at least one of an endothelial cell (EC), a CD34⁺CD31⁺ cell, or a CD34⁺CD144⁺ cell.

3. The method of claim 1, wherein hematopoietic progenitor cell comprises at least one of a CD34⁺CD43⁺ cell and a CD34⁺CD45⁺ cell.

4. The method of claim 1, wherein the hemato-lymphoid cell comprises a conventional natural killer (cNK) cell.

5. The method of claim 1, wherein disrupting AHR activity comprises treating the cell with an aryl hydrocarbon receptor (AHR) antagonist.

6. The method of claim 5, wherein the AHR antagonist comprises StemReginin-1 (SR-1).

7. The method of claim 1, wherein disrupting AHR activity comprises reducing expression of AHR in the cell.

8. The method of claim 7, wherein the reduction of expression of AHR is inducible.

9. The method of claim 1, wherein the stem cell is an embryonic stem cell or an induced pluripotent stem cell (iPSC).

10. The method of claim 1, wherein the method further comprises treating the cell with media comprising at least one of IL-15, IL-7, Flt-3 ligand, stem cell factor, and IL-3.

11. A hemato-endothelial cell, a hematopoietic progenitor cell, or a hemato-lymphoid cell derived from the methods according to claim 1.

12. A method comprising
inducing hematopoietic differentiation of a human stem cell; and
enhancing aryl hydrocarbon receptor (AHR) activity in the cell,
wherein differentiation of the cell to an innate lymphoid cell group 3 (ILC3) cell is enhanced compared to differentiation without enhancement of AHR activity.

13. The method of claim 12, wherein the ILC3 cell is at least one of CD94⁻, CD117⁺, CD56⁺, and LFA1⁻.

14. The method of claim 12, wherein enhancing AHR activity comprises treating the cell with an AHR agonist.

15. The method of claim 14, wherein the AHR agonist comprises tetrachlorodibenzo-p-dioxin (TCDD).

16. The method of claim 12, wherein enhancing AHR activity comprises increasing expression of AHR in the cell.

17. The method of claim 12, wherein the enhancement is inducible.

18. The method of claim 12, wherein the stem cell is an embryonic stem cell or an induced pluripotent stem cell (iPSC).

19. The method of claim 12, wherein the method further comprises treating the cell with media comprising at least one of IL-15, IL-7, Flt-3 ligand, stem cell factor, and IL-3.

20. A ILC3 cell derived from the method according to claim 12.


SEQUENCE LISTING (45 sequences)

SEQ ID NO: 1 - DNA, 19 bp, artificial
  primer
tcggccacggagtttcttc

SEQ ID NO: 2 - DNA, 20 bp, artificial
  primer
ggtcagcatgtgcccaatca

SEQ ID NO: 3 - DNA, 20 bp, artificial
  primer
acatgctgaccctgggaaag

SEQ ID NO: 4 - DNA, 19 bp, artificial
  primer
ggtgtggagccaattcgga

SEQ ID NO: 5 - DNA, 19 bp, artificial
  primer
tgagtgccgtgtgtttcgg

SEQ ID NO: 6 - DNA, 21 bp, artificial
  primer
gttgctgaagttgcggttgag

SEQ ID NO: 7 - DNA, 19 bp, artificial
  primer
ccactcctccacctttgac

SEQ ID NO: 8 - DNA, 18 bp, artificial
  primer
accctgttgctgtagcca

SEQ ID NO: 9 - DNA, 20 bp, artificial
  guide RNA
tcagattgtccctggaggtc

SEQ ID NO: 10 - DNA, 18 bp, artificial
  primer
aggcagctcacctgtact

SEQ ID NO: 11 - DNA, 23 bp, artificial
  primer
catctcgccttaccaaactctac

SEQ ID NO: 12 - DNA, 21 bp, artificial
  primer
cttaggctcagcgtcagttac

SEQ ID NO: 13 - DNA, 23 bp, artificial
  primer
cgtttctttcagtaggggaggat

SEQ ID NO: 14 - DNA, 19 bp, artificial
  primer
tcggccacggagtttcttc

SEQ ID NO: 15 - DNA, 20 bp, artificial
  primer
ggtcagcatgtgcccaatca

SEQ ID NO: 16 - DNA, 21 bp, artificial
  primer
aagttcttgaggcactgcgaa

SEQ ID NO: 17 - DNA, 20 bp, artificial
  primer
ggccggtacgttctccaaat

SEQ ID NO: 18 - DNA, 18 bp, artificial
  primer
gcagctcggaaggcagat

SEQ ID NO: 19 - DNA, 25 bp, artificial
  primer
tggattttaaaagcgagaagacttg

SEQ ID NO: 20 - DNA, 27 bp, artificial
  primer
gtcacaaattgactgttacaacaccat

SEQ ID NO: 21 - DNA, 27 bp, artificial
  primer
ttctactagatgagagggtgtctgagg

SEQ ID NO: 22 - DNA, 19 bp, artificial
  primer
gggatcacactgagcttgc

SEQ ID NO: 23 - DNA, 19 bp, artificial
  primer
acccctgattctggtgtgg

SEQ ID NO: 24 - DNA, 19 bp, artificial
  primer
gcgtctccagcctcatctt

SEQ ID NO: 25 - DNA, 20 bp, artificial
  primer
ggaagagtccgctgctgtag

SEQ ID NO: 26 - DNA, 20 bp, artificial
  primer
cacagcgagttcgagagctt

SEQ ID NO: 27 - DNA, 20 bp, artificial
  primer
gatgggtactggaggcacat

SEQ ID NO: 28 - DNA, 19 bp, artificial
  primer
gcagagccgatggaattag

SEQ ID NO: 29 - DNA, 20 bp, artificial
  primer
atcagtttccgacgttctca

SEQ ID NO: 30 - DNA, 20 bp, artificial
  primer
tcagattgtccctggaggtc

SEQ ID NO: 31 - DNA, 20 bp, artificial
  primer
agtttgttggtcccaggttg

SEQ ID NO: 32 - DNA, 20 bp, artificial
  primer
cggatatcagcatcctgtcc

SEQ ID NO: 33 - DNA, 21 bp, artificial
  primer
tcatgaacaccgcttattcag

SEQ ID NO: 34 - DNA, 22 bp, artificial
  primer
gatgtttgtggacgtggtcttg

SEQ ID NO: 35 - DNA, 21 bp, artificial
  primer
ctttccacactgcacccactt

SEQ ID NO: 36 - DNA, 19 bp, artificial
  primer
gcccctcattaagcccaag

SEQ ID NO: 37 - DNA, 21 bp, artificial
  primer
ttgtggtggtctgacagttcg

SEQ ID NO: 38 - DNA, 21 bp, artificial
  primer
gccaaggctcagtcatgagaa

SEQ ID NO: 39 - DNA, 20 bp, artificial
  primer
ttgtccccacagattttgca

SEQ ID NO: 40 - DNA, 25 bp, artificial
  primer
cctgctatgattttctcccaataaa

SEQ ID NO: 41 - DNA, 30 bp, artificial
  primer
aacacaaaaatatcacagtcagaggtagac

SEQ ID NO: 42 - DNA, 21 bp, artificial
  primer
gcttgacaagtccaacttcca

SEQ ID NO: 43 - DNA, 22 bp, artificial
  primer
gctcactcatactgactccgtg

SEQ ID NO: 44 - DNA, 19 bp, artificial
  primer
ccactcctccacctttgac

SEQ ID NO: 45 - DNA, 18 bp, artificial
  primer
accctgttgctgtagcca